(19) United States
(12) Patent Application Publication
(10) Pub. No.: US 2021/0340562 A1
(43) Pub. Date: Nov. 4, 2021

(54) ENGINEERED LONG INTERSPERSED ELEMENT (LINE) TRANSPOSONS AND METHODS OF USE

(71) Applicant: Board of Regents, The University of Texas System, Austin, TX (US)

(72) Inventor: Shawn Christensen, Arlington, TX (US)

(21) Appl. No.: 17/286,772

(22) PCT Filed: Oct. 21, 2019
(86) PCT No.: PCT/US2019/057244

§ 371 Date: Apr. 19, 2021

Related U.S. Application Data
(60) Provisional application No. 62/748,227, filed on Oct. 19, 2018.

(51) Int. Cl. C12N 15/85; C12N 9/12; C12N 9/22

(52) U.S. Cl. CPC C12N 15/85; C12N 9/1276; C12N 2800/90; C12N 9/22; C12N 2840/203; C12Y 207/07049

(57) ABSTRACT

Engineered transposons and methods of use thereof are provided. The transposons typically include an RNA component and a protein component. The RNA component can include, for example, a DNA targeting sequence, one or more protein binding motifs, and a nucleic acid sequence of interest to be integrated into a target DNA. The protein component is typically derived from a RLE LINE element protein and can include a DNA binding domain, an RNA binding domain, a reverse transcriptase, a linker domain, and an endonuclease. Pharmaceutical compositions and methods of use for introducing nucleic acid sequences into the genomes of cells are also provided.

### CROSS-REFERENCE TO RELATED APPLICATIONS

This application claims the benefit of and priority to U.S. Ser. No. 62/748,227 filed Oct. 19, 2018, which is hereby incorporated by reference in its entirety.

### STATEMENT REGARDING FEDERALLY SPONSORED RESEARCH

This invention was made with government support under grant 0950983 awarded by the National Science Foundation. The government has certain rights in the invention.

### REFERENCE TO THE SEQUENCE LISTING

The Sequence Listing submitted as a text file named “UTSB_18_47_PCT_ST25.txt,” having a size of 17,183 bytes is hereby incorporated by reference pursuant to 37 C.F.R. § 1.52(e)(5).

### FIELD OF THE INVENTION

The invention is generally drawn to compositions and methods for genome modification.

### BACKGROUND OF THE INVENTION

Genome editing technologies have therapeutic potential for various diseases and disorders including, but not limited to, cancer, genetic disorders, and HIV/AIDS. Genome editing of somatic cells is a promising area of therapeutic development, and the complex enzyme-editing tool CRISPR-Cas9 has been used to eliminate the human β-globulin (HBB) gene from the germline of human embryos (Otieno, (2015), J Clin Res Bioeth 6:253. doi: 10.4172/2155-9627.1000253). However, historically, the clinical application of gene editing technology has been limited by, among other concerns, low frequency of editing events, high off-target events, or a combination thereof.

Thus, it is an object of the invention to provide improved compositions and methods for gene delivery and gene editing.

### SUMMARY OF THE INVENTION

Engineered transposons and methods of use thereof are provided. The transposons typically include a RNA component and a protein component. The RNA component can include, for example, a DNA targeting sequence, one or more protein binding motifs, and a nucleic acid sequence of interest to be integrated at a DNA target site. The DNA targeting sequence, the protein binding motifs, and sequence of interest are typically operably linked such that they can bind to a protein component derived from a Restriction-like Endonuclease Long Interspersed (RLE LINE) element protein and be reverse transcribed, and the resulting cDNA can be integrated into the DNA at the DNA target site, for example in a cellular genome. The sequence of interest can encode, for example, a gene or a fragment thereof, or a functional nucleic acid.

The RNA segments involved in binding to protein, the protein binding motifs (PMB), typically bind to an RNA binding domain (domain −1), a reverse transcriptase, a linker domain, an endonuclease, or a combination thereof of the protein component.

The RNA component can include elements from or derived from a parental LINE or SINE backbone and the nucleic acid sequence of interest of RNA component is typically heterologous to the LINE or SINE. In typical embodiments, the DNA targeting sequence is heterologous to the parental LINE or SINE. The RNA component can include for example, 3′ PBM sequence from or derived from a parental LINE or SINE element, a CRISPR/Cas tracer sequence, a CRISPR/Cas guide sequence, or a combination thereof, a 5′ PBM sequence from or derived from the parental LINE or SINE element, preferably wherein any IRES sequence is non-functional, a ribozyme such as Hepatitis Delta Virus like ribozyme, or any combination thereof.

The protein component is typically derived from a RLE LINE element protein and can include one or more DNA binding domains, one or more RNA binding domains, a reverse transcriptase, a linker domain, and an endonuclease. Typically, the DNA binding domains, RNA binding domains, reverse transcriptase, linker domain, and endonuclease are operably linked such that they can bind to an RNA component and DNA (e.g., cellular genomic DNA) at the DNA target site, and facilitate reverse transcription of the RNA component into cDNA, and integration of the cDNA into the DNA at the DNA target site. Typically, the DNA binding domain is mutated relative to the parental LINE DNA binding domain, or the parental DNA binding domain is substituted with an alternative DNA binding domain. In some embodiments, the DNA binding domain is a DNA binding domain from another DNA binding protein, or a motif thereof such as a helix-turn-helix, zinc finger, leucine zipper, winged helix, winged helix-turn-helix, helix-loop-helix, HMG-box, Wor3 domain, OB-fold domain, immunoglobulin fold, B3 domain, TAL effector, or RNA-guided domain. Typically, the sequences of one or more of the RNA binding domain, reverse transcriptase, linker domain, and endonuclease are the same as those of the LINE element protein, or preferably mutated to improve binding and/or enzymatic activity for the RNA component or target DNA relative to the parental LINE element protein.

In some embodiments, the parental LINE or SINE backbone of the RNA component and the parental LINE backbone of the protein component are the same LINE and/or the SINE is derived from or an ancestor of the LINE The RNA sequence of the RNA component, the amino acid sequence of the protein sequence, or a combination thereof can be recombinant sequences and/or variants of the parental backbones.

Vectors encoding the RNA component and the protein component, as well as pharmaceutical compositions including the components, the vectors, and/or the engineered transposons formed therefrom are also provided. Preferably the transposons can form a productive 4-way junction during the integration reaction at the DNA target site.

Methods of use are also provided. For example, a method of introducing a nucleic acid sequence of interest into the genome of a cell or cells can include contacting the cell or cells with (i) an RNA component or a vector encoding the RNA component in combination with a protein component or a vector encoding the protein component; or (ii) the engineered transposon including both the RNA and protein components. The cells can be contacted in vitro or in vivo. In some embodiments, ex vivo modified cells are subsequently introduced into a subject in need thereof. In some embodiments, the compositions are administered directly to the subject in need thereof.

Methods of treating diseases and disorders are also provided. In such uses, expression of the nucleic acid sequence of interest in the cells can improve one or more symptoms of a disease or disorder, or a molecular pathway underlying a disease or disorder. In preferred embodiments, an effective number of cells are modified to treat a subject with the disease or disorder.

### BRIEF DESCRIPTION OF THE DRAWINGS

FIG. 1A is a cartoon diagram of a R2Bm structure. R2Bm RNA (wavy line) and open reading frame (ORF) structure (box). The ORF encodes conserved domains of known and unknown functions: zinc finger (ZF), Myb (Myb), reverse transcriptase domain (RT), a cysteine-histidine rich motif (CCHC), and a PD-(D/E)XK type restriction-like endonuclease (RLE). RNA structures present in the 5′ and 3′ untranslated regions that bind R2 protein are marked as 5′ and 3′ protein binding motifs (PBMs), respectively. Brackets indicate the individual segments of the R2Bm RNA used in this paper: 5′ PBM RNA (320 nt), 3′ PBM RNA (249 nt), RNA at the 5′ end of the element (25 or 40 nt) and RNA 3′ end (25 or 40 nt). FIG. 1B is a cartoon diagram of a R2Bm integration reaction. The four-step integration model is depicted on a segment of 28S rDNA (parallel lines). An R2 protein subunit (hexagon) is bound upstream of the insertion site (vertical bar) and an R2 protein subunit is bound downstream of the insertion site. The upstream subunit is associated with the 3′ PBM RNA while the downstream subunit is associated with the 5′ PBM RNA. The footprint of the protein subunits on the target DNA are indicated. The upstream footprints from −40 bp to −20 bp, but grows to just over the insertion site (vertical line) after first-strand DNA cleavage. The downstream subunit footprints from just prior to the insertion site to +20 bp (Christensen, et al., Nucleic Acids Res 33, 6461 (2005), Christensen and Eickbush, Proc Natl Acad Sci USA 103, 17602 (2006)). The four steps of integration are: (1) DNA cleavage of the bottom/first-strand of the target DNA, (2) TPRT, (3) DNA cleavage of the top/second-strand of the target DNA, and (4) second strand DNA synthesis. The fourth step not previously been directly observed in vitro. The overlapping portions of the target site used in Examples 1-8 are indicated with brackets.

FIGS. 2A and 2B are diagrams of the nonspecific 4-way junction (2A) and linear DNA (2B) DNA constructs. The design and sequence of the 4-way junction was from (Middleton and Bond, Nucleic Acids Res 32, 5442 (2004)) and formed by annealing the b, x, h, and r DNA oligos. Each arm of the resulting junction was 25 bp. The linear DNA was generated by annealing oligo b to an oligo that was a combination of the x and h oligos. Thus junction and linear DNAs shared a common DNA oligo (oligo b). The shared DNA oligo was 5′ end-labeled (star) with 32P prior to formation and purification of the linear and junction DNAs.

FIG. 3 is a diagram of several linear, 3-way, and 4-way branched DNA constructs. Straight lines represent DNA and wavy lines represent RNA. Thin lines represent non-specific DNA depicted in FIG. 2A-2B. Thick lines represent 28S rDNA as well as R2 element derived sequences. The R2 sequences are from the 5′ and 3′ ends of the element. The 28S sequence is the downstream DNA (28Sd) plus 7 bp of upstream DNA. The “arms” in each construct are 25 bp in length. Each construct is numbered for discussion purposes. The star indicates that the strand was end labelled as in previous figures. Two variations of construct v were tested, one having a DNA duplex in the R2 3′ arm and the other having the RNA/DNA hybrid that would have been the result of TPRT. No detectable second-strand DNA cleavage was found on constructs i-v. Second-strand DNA cleavage was detectable on constructs vi-viii.

FIG. 4A is a diagram of several derivatives of the 4-way junction from FIG. 3 to test for cleavage on partial junctions. The constructs have been numbered. The 28S downstream (28Sd) DNA arm was increased 47 bp so as to equal to the amount of downstream DNA historically used in a linear 28S target DNA (Christensen, et al., Nucleic Acids Res 33, 6461 (2005), Christensen and Eickbush, Proc Natl Acad Sci USA 103, 17602 (2006)). FIG. 4B is a graph of the fraction cleaved (f cleaved) as a function of the fraction bound (f bound) for each set of the constructs of FIG. 4A. Diameter of the dot depicts relative cleavability of the construct by R2Bm. FIG. 4C is a diagram of constructs designed to test DNA cleavage on 4-way junctions that include upstream 28S DNA. The 28S upstream (28Su) DNA arm is 73 bp and corresponds to the amount of upstream DNA normally used in a linear target DNA (Christensen and Eickbush, Mol Cell Biol 25, 6617 (2005), Christensen and Eickbush, J Mol Biol 336, 1035 (2004)). Black lines are DNA with thin lines being non-specific DNA and thick line being either 28S or R2 derived DNA. FIG. 4D is a graph of the fraction cleaved (f cleaved) as a function of the fraction bound (f bound) for each set of the constructs of FIG. 4C. Diameter of the dot depicts relative cleavability of the construct by R2Bm. Abbreviations and symbols are as in previous figures.

FIG. 5 is a diagram of the 4-way junction for denaturing gel analysis of DNA cleavage (-dNTP) and cleavage plus second-strand synthesis (+dNTP) reactions.

FIG. 6A is a diagram of constructs designed to hold the pre-cleaved products close proximity and to test which arm is use as a template. The length of 5′ and 3′ arms were varied (40 bp vs 25 bp). The 28S downstream arm was 47 bp and the 28S upstream arm was 73 bp. FIG. 6B is a diagram of constructs designed to test whether the upstream or the downstream protein subunit is likely responsible for second strand synthesis. FIG. 6C is a graph of the fraction synthesized (f synthesized) as a function of the fraction bound (f bound).

FIG. 7A is a diagram showing a new model for R2 integration. The R2 28S target site is labelled with the positions of the first and second-strand cleavages that will lead to insertion of a R2 new element. The initial steps of the integration reaction (I, ii) are as in FIG. 1B except that the target site is bent 90° near the second strand insertion site for diagrammatic purposes. Step iii depicts a template jump/recombination event near the second-strand cleavage site that generates the 4-way junction. Step iv depicts second-strand cleavage. Finally, step v depicts second-strand DNA synthesis. Abbreviations: up (target sequences upstream of the insertion site), dwn (target sequences downstream of the insertion site). FIG. 7B is a diagram showing a new model for L1 integration. A target site is labelled with the first and second-strand cleavages staggered such that a target site duplication (tsd) would occur upon element insertion. The steps are as in R2 except that the template jump displaces/melts the tsd region of the target to generate the 4-way junction.

FIG. 8A is a cartoon diagram showing an R2 target site, 28S rDNA, and insertion model. R2 protein associated with the 3′ PBM RNA binds 20 to 40 bases upstream (28Su) of the insertion site (vertical line) and protein associated with the 5′ PBM RNA binds to 20 bases downstream of the insertion site (Christensen, et al., Nucleic Acids Res. 33, 6461-6468 (2005), Christensen and Eickbush, J. Mol. Biol. 336, 1035-1045 (2004)). Insertion occurs in five steps: (1) First strand cleavage by upstream protein subunit endonuclease. (2) First strand synthesis (TPRT) by the upstream protein subunit reverse transcriptase. (3) Template jump/recombination to upstream target DNA (28Su) resulting in a four-way junction branched structure (zoomed in diagram). (4) Second strand cleavage by endonuclease of the downstream protein subunit. (5) Second strand synthesis by reverse transcriptase of downstream protein subunit. FIG. 8B is a multiple sequence and secondary structure alignment of the linker region of RLE LINEs (SEQ ID NO:31-44). Stars represent the residues that were mutated and half triangle represents double point mutants generated in the presumptive α-finger and the zinc knuckle regions. Double point mutants generated for this study were: GR/AD/A, H/AIN/AALP, SR/AIR/A, SR/AGR/A, C/SC/SHC, CR/AAGCK/A, HILQ/AQ/A and RT/AH/A. The first four mutants are in the presumptive α-finger region and the last four mutants are in the zinc knuckle region as indicated by the brackets on the top. Secondary structures are predicted by Ali2D and grey bars represent α-helices and arrow represents β-strands. Abbreviations: R2Bm=Bombyx mori, R2Dm=Drosophila melanogaster, R2Dana=Drosophila ananassae, R2Dwil=Drosophila willistoni, R2Dsim=Drosophila simulans, R2Dpse=Drosophila pseudoobscura, R2Fauric=Forficula auricularia, R2Amar=Anurida maritima, R2Nv-B=Nasonia vitripennis, R2Lp=Limulus polyphemus, R2Amel=Apis mellifera, R2Dr=Danio rerio, R8Hm-A=Hydra magnipapillata, R9Av-1=Adineta vaga.

FIGS. 9A and 9B are bar graphs reporting mutant's ability to bind to target DNA in the presence of 3′ (9A) and 5′ PBM (9B) RNAs. Wild type (WT) protein activity is set to 1 and the mutant protein activity is then given as a fraction of WT activity (fWTactivity). The bars for each graph represent, left-to-right: R2: WT, H/AIN/AALP, C/SC, SHC.

FIG. 10A-10D are bar graphs showing DNA binding by α-finger mutant proteins. FIGS. 10A and 10B report the relative ability of the mutants to bind to linear target DNA. WT and KPD/A WT served as positive controls while Pet28a and DNA only lanes served as negative controls. Standard deviation is presented on top of the bars. FIG. 10C reports the binding to an analog of the branched insertion intermediate. The star in the substrate diagrams indicates the strand that was 5′end labelled. FIG. 10D reports the linear target DNA binding activity of α-finger mutant proteins in the absence of RNA. The bars for each graph represent, left-to-right: R2: KPD/A WT, GR/AD/A, SR/AIR/A, SR/AGR/A.

FIG. 11 is a scatter plot showing first strand DNA cleavage activity by α-finger mutant proteins. The fraction of target DNA that undergoes first strand cleavage (fcleaved) was quantitated from a denaturing gel. The scatter plot shows the fraction of cleaved target DNA (fcleaved) plotted as a function of fraction of target DNA bound by protein (fbound) at each protein concentrations. Data points for WT, GR/AD/A, SR/AIR/A and SR/AGR/A are represented by asterisk, white box, grey box, and black box respectively.

FIG. 12A is an illustration of the experimental setup for first strand synthesis assay in which pre-cleaved target DNA was incubated with R2 protein in the presence of 3′ PBM RNA and dNTPs. FIG. 12B is a scatter plot showing the fraction of the DNA that underwent synthesis (fsynthesis) as a function of fraction of the DNA that was bound by R2 protein (fbound) across a protein titration series. The symbols and abbreviations are as in the previous figures.

FIG. 13A is a scatter plot of second strand cleavage activity by α-finger mutant proteins on linear target DNA. An EMSA gel was used to calculate the fraction of target DNA bound by R2 protein. A denaturing gel was used to calculate the fraction of target DNA cleaved by the R2 protein. Symbols and abbreviations are as in previous figures. FIG. 13B is a scatter plot of second strand DNA cleavage activity by α-finger mutant proteins on four-way junction DNA. An EMSA gel used to calculate the fraction of target DNA bound by the R2 protein. A denaturing gel used to calculate the fraction of target DNA cleaved by the R2 protein. Symbols and abbreviations are as in previous figures.

FIG. 14A is a diagram illustrating the experimental setup for a second strand synthesis assay in which pre-cleaved four-way junction DNA was incubated with R2 protein in the presence of dNTPs. FIG. 14B Scatter plot of second strand synthesis activity. Symbols and abbreviations are as in previous figures.

FIG. 15A is a scatter plot showing first strand cleavage activity of zinc knuckle mutant proteins. The fraction of cleaved target DNA (fcleaved) is plotted as a function of the fraction of target DNA bound by protein (fbound) at each protein concentrations. FIG. 15B is a scatter plot showing first strand synthesis activity of zinc knuckle mutant proteins. The graph plots fraction of target DNA that undergoes first strand synthesis by TPRT (fsynthesis) as a function of fraction of pre-cleaved linear target DNA bound by the protein (fbound). FIG. 15C is a scatter plot showing second strand cleavage activity of zinc knuckle mutants on a 4-way junction target DNA. The graph plots target DNA cleaved at the second strand (fcleaved) as a function of fraction of 4-way junction DNA bound by the protein (fbound). FIG. 15D is a scatterplot of second strand cleavage activity by zinc knuckle mutants on linear target DNA as a function bound DNA.

FIG. 16 is a scatter plot of second strand synthesis activity of zinc knuckle mutants. Experimental setup was as in FIG. 14A.

FIG. 17A is a series of domain maps showing ORF structure of R2Bm, human L1 (L1Hs), and Saccharomyces cerevisiae Prp8 (Mahbub, et al., Mob. DNA 8, 1-15 (2017), Wan, et al., Science (80-.) (2016) doi:10.1126/science.aad6466; Bertram, et al., Cell (2017); doi:10.1016/j.cell.2017.07.011; Qu, et al., Nat. Struct. Mol. Biol. (2016); doi:10.1038/nsmb.3220; Nguyen, et al., Nature 530, 298-302 (2016); Galej, et al., Current Opinion in Structural Biology (2014). doi:10.1016/j.sbi.2013.12.002; Blocker, et al., RNA 11, 14-28 (2005)). In the linker region, the sequences of the α-helices (rounded bars) with an asterisk align well. Remaining of the colored α-helix and β-strands (arrows) (may) form a structurally similar knuckle. FIG. 17B is a model of R2Bm's RT and RLE (Mahbub, et al., Mob. DNA 8, 1-15 (2017)). FIG. 17C is a cryo-Em structure of the large fragment of Prp8 (Wan, et al., Science (80-.). (2016). doi:10.1126/science.aad6466). FIG. 17D is a cryo-EM structure of the Prp8 and RNA from the B spliceosome complex (Bertram, et al., Cell (2017). doi:10.1016/j.cell.2017.07.011). A branched structure formed by the RNA components of spliceosome is also shown.

FIG. 18A is a diagram of the RNA components of an engineered LINE. HDV=hepatitis delta virus ribozyme (optional); PBM=protein binding motifs (can be from one element or from two elements if forming a hetero RNP); Prom=pol II promotor and related transcription factor binding sites for ORF expression; ORF=ORF of gene being brought into the genome via TPRT; tracr=tracer RNA; tracr/guide=standard cas 9 targeting RNA; TS=Target Sequence. Tracer, guide, or tracer/guide can be supplied in cis (as above) or in trans. FIG. 18B is a diagram of an RLE ORF with engineered DNA binding domain. R2 or other RLE protein expression construct can be expressed in bacteria (in order to be purified for use) or eukaryotic expression system for direct production in the intended cells. Engineered DB=ZF from ZF library, or talens, or cas9 (EN-) Note: DB in R2 is ZFs and Myb. αF=α-Finger. FIG. 18C is a diagram of two different models of RLE LINE binding at the target site. FIG. 18D is a diagram of two different models of RLE LINE integration.

### DETAILED DESCRIPTION OF THE INVENTION

### I. Definitions

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, the term “construct” refers to a recombinant genetic molecule having one or more isolated polynucleotide sequences.

As used herein, the term “regulatory sequence” refers to a nucleic acid sequence that controls and regulates the function, for example, transcription and/or translation of another nucleic acid sequence. Control sequences that are suitable for prokaryotes, may include a promoter, optionally an operator sequence and/or a ribosome binding site. Eukaryotic cells are known to utilize sequences such as promoters, terminators, polyadenylation signals, and enhancers. Regulatory sequences include viral protein recognition elements that control transcription and replication of viral genes.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory sequences and can include 5′ and 3′ ends.

As used herein, the term polypeptide includes proteins and fragments thereof. The polypeptides can be “endogenous,” or “exogenous,” meaning that they are “heterologous,” i.e., foreign to the host cell being utilized, such as human polypeptide produced by a bacterial cell. Polypeptides are disclosed herein as amino acid residue sequences.

As used herein, the term “vector” refers to a replicon, such as a plasmid, phage, or cosmid, into which another DNA segment may be inserted so as to bring about the replication of the inserted segment. The vectors can be expression vectors.

As used herein, the term “expression vector” refers to a vector that includes one or more expression control sequences.”

As used herein, the terms “transfected” or “transduced” refer to a host cell or organism into which a heterologous nucleic acid molecule has been introduced. The nucleic acid molecule can be stably integrated into the genome of the host or the nucleic acid molecule can also be present as a stable or unstable extrachromosomal structure. Such an extrachromosomal structure can be auto-replicating. Transformed cells or organism may to encompass not only the end product of a transformation process, but also transgenic progeny thereof. A “non-transformed,” or “non-transduced” host refers to a cell or organism, which does not contain the heterologous nucleic acid molecule.

As used herein, the term “endogenous” with regard to a nucleic acid refers to nucleic acids normally present in the host.

As used herein, the term “heterologous” refers to elements occurring where they are not normally found. For example, an endogenous promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompass “exogenous” and “non-native” elements.

### II. Engineered Transposons

Long interspersed elements (LINEs) are an abundant and diverse group of autonomous transposable elements (TEs) that are found in eukaryotic genomes across the tree of life. LINEs also mobilize the non-autonomous short interspersed elements (SINEs). SINEs appropriate the protein machinery of LINEs to replicate. The movement of LINEs and SINEs have been implicated in progression to cancer and in genome evolution including modulation of gene expression, genome rearrangements, DNA repair, and as a source of new genes. LINEs replicate by a process called target primed reverse transcription (TPRT) where the element RNA is reverse transcribed into DNA at the site of insertion using a nick in the target DNA to prime reverse transcription (Luan, et al., Cell 72, 595 (1993); Cost, et al., EMBO J 21, 5899 (2002); Moran, et al., Eds. (ASM Press, Washington, D.C., 2002), pp. 836-869). LINEs encode protein(s) that are used to perform the important steps of the insertion reaction. LINE proteins bind their own mRNA, recognize target DNA, perform first-strand target-DNA cleavage, and perform TPRT. The proteins are also believed to perform second-strand target-DNA cleavage and second-strand element-DNA synthesis, although the evidence for this is sparse (Luan, et al., Cell 72, 595 (1993); Cost, et al., EMBO J 21, 5899 (2002); Moran, et al., Eds. (ASM Press, Washington, D.C., 2002), pp. 836-869; Christensen and Eickbush, Mol Cell Biol 25, 6617 (2005); Kulpa and Moran, Nat Struct Mol Biol 13, 655 (2006); Dewannieux and Heidmann, Cytogenet Genome Res 110, 35 (2005); Doucet, et al. Mol Cell 60, 728 (2015); Christensen, et al., Nucleic Acids Res 33, 6461 (2005); Govindaraju, et al., Nucleic Acids Res 44, 3276 (2016); Martin, RNA Biol 7, 67 (2010); Martin, J Biomed Biotechnol 2006, 45621 (2006); Matsumoto, et al., Mol Cell Biol 26, 5168 (2006); Zingler et al., Genome Res 15, 780 (2005); Kurzynska-Kokorniak, et al., J Mol Biol 374, 322 (2007); Ichiyanagi, et al. N. Okada, Genome Res 17, 33 (2007); Gasior, et al., J Mol Biol 357, 1383 (2006); Suzuki et al., PLoS Genet 5, e1000461 (2009); Christensen and Eickbush, Proc Natl Acad Sci USA 103, 17602 (2006)).

The early branching clades of LINEs encode a restriction-like endonuclease (RLE) while the later branching LINEs encode an apurinic-apyrimidinic DNA endonuclease (APE) (Eickbush and Malik, in Origins and Evolution of Retrotransposons, Craig, N L, Craigie, R, Gellert, M, A. M. Lambowitz, Eds. (ASM Press, Washington, D.C., 2002), pp. 1111-1146; Yang, et al., Proc Natl Acad Sci USA 96, 7847 (1999); Feng, et al., Cell 87, 905 (1996); Weichenrieder, et al., Structure 12, 975 (2004)). Both types of elements are thought to integrate through a functionally equivalent integration process (Moran, et al., Eds. (ASM Press, Washington, D.C., 2002), pp. 836-869; Han, Mob DNA 1, 15 (2010); Fujiwara, Microbiol Spectr 3, MDNA3 (2015); Eickbush and Eickbush, Microbiol Spectr 3, MDNA3 (2015)).

Replication occurs through an ordered series of DNA cleavage and polymerization events using encoded nucleic acid binding, endonuclease, and polymerase functions (Christensen and Eickbush, Proc Natl Acad Sci USA 103, 17602 (2006); Shivram, et al., Mobile Genetic Elements, 1:3, 169-178 (2011), see also the Examples below). The element encoded protein(s), once translated, form a ribonucleoprotein (RNP) particle with the transcript from which they were translated—a process called cis-preference. The RNP binds to the target DNA, cuts one of the DNA strands, and uses the target site's exposed 3′-OH to prime reverse transcription of the element RNA into cDNA (cDNA)—a process called target primed reverse transcription (TPRT). The opposing target DNA strand is then cleaved. The cDNA is turned into double stranded DNA, completing the integration event. Successful integration of the newly reverse transcribed DNA at a target site depends on interplay between the DNA, RNA, and protein components of the transposon and the target site DNA.

Engineered RNA components and protein components that utilize sequences and mechanisms from, or derived from, LINE and SINE retrotransposons, and engineered transposons formed therefrom are provided. As used herein, to be “derived” from a LINE or SINE means that the RNA and/or the protein component can trace the origin of one or more of its domains to a corresponding RNA or protein component of a parental LINE or SINE. In some embodiments, the engineered RNA or protein component has one or more domains deleted, substituted, added, or mutated relative to the corresponding RNA or protein component of a parental LINE or SINE. In some embodiments, the engineered RNA and/or protein component has at least 50, 60, 70, 75, 80, 85, 80, 95 or more percent sequence identity to the nucleic acid or amino acid sequence of a corresponding RNA or protein component of a parental LINE or SINE. The engineered RNA and/or the protein component can include sequences, including entire domains, that are heterologous to a corresponding RNA or protein component of a parental LINE or SINE. The engineered RNA and/or the protein components can be recombinant sequences.

Typically, an RNA component containing a gene of interest to be inserted/delivered into the genome can be bound to the engineered protein component. The RNA is converted into DNA and inserted into the genome by Target Primed Reverse Transcription (first strand DNA cleavage, priming of cDNA from liberated target site 3-OH, second strand cleavage, second strand synthesis) mediated by the protein component.

In order to change the site of insertion, the existing DNA binding regions of RLE LINEs including the amino-terminal ZFs/myb, the Linker's α-finger (see the Examples below), and the RLE (Govindaraju, et al., Nucleic Acids Res 44, 3276 (2016)), can be modified or replaced to bind and cleave new sites of interest. The ZFs/myb are candidates to be replaced with DNA binding domains that target new sites of interest. In some embodiments, the linker, RT, RLE can generally be modified in place. Different RLE LINE backbones can be used and swapped in whole and in part. Possible sources of DNA binding modules to use for the amino-terminal domain include, zinc fingers from a zinc finger library, Talens, CRISPR/cas, and others as discussed in more detail below.

When altering the transposon's coding and non-coding nucleic acid sequences to engineer a re-targeted gene delivery system, steps should be taken to ensure that each of the component parts of the system remains structurally and functionally compatible while also specifically targeting the desired site (e.g., genomic location). Design considerations for important structural elements are discussed in more detail below. Regardless of the component parts selected by the practitioner, care should be taken to ensure the engineered transposon can carry out the basic activities to integrate: RNA binding activity, DNA binding activity, DNA endonuclease activity, reverse transcriptase (RT) activity, and completion of integration by second strand synthesis.

A. Structure of the Engineered Transposons

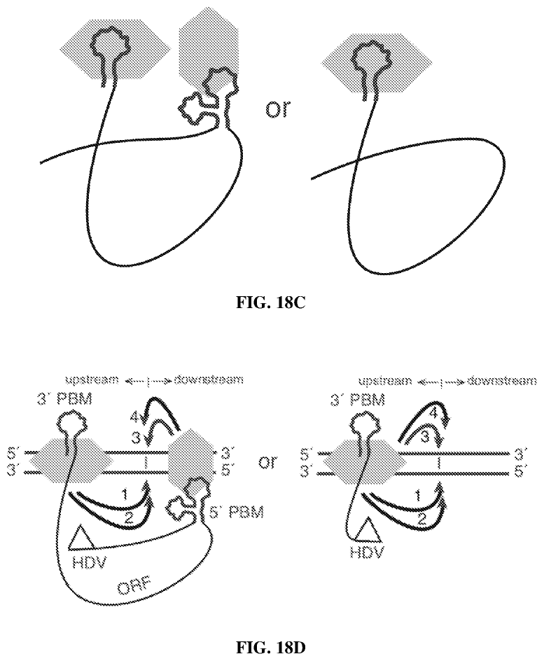

An exemplary engineered transposon-based on RLE LINE backbone is outlined in FIGS. 18A-18D. The engineered transposon includes an RNA component and protein component.

1. RNA Component

Generally, the RNA component includes element(s) that allow for or facilitate binding of the protein component to the RNA component, element(s) that allow for or facilitate targeting, preferably binding (e.g., priming), of the engineered transposon to the DNA target site, and elements(s) that allow for or facilitate one or more of the endonuclease, reverse transcription, and integration activities of the protein component or other endonucleases, reverse transcriptases, or accessory elements provided in trans. At a minimum, the design of the RNA component, including both the primary and secondary structure thereof, should not prevent, and preferably aids, in the proper integration of the open reading frame of interest into the DNA target site.

An exemplary RNA component of the engineered transposons is illustrated in FIG. 18A. Thus, for example, the RNA component of the engineered transposon can include one or more of a target sequence (TS), a ribozyme (e.g., hepatitis delta virus ribozyme) (HDV), a tracr sequence (e.g., tracr, guide, or tracr/guide sequence, e.g., Cas9 targeting RNA)), a sequence encoding a IRES/PBM protein binding motif domain, a promoter (e.g., a pol II promoter or transcription factor binding sites to ensure ORF expression) (Prom), an open reading frame (ORF) encoding a transgene of interest for insertion at the target site, and PBM protein binding motif. The tracer, guide, or tracer/guide can be supplied in cis or in trans. The RNA component need not, and preferably does not, include a sequence encoding the open reading from a LINE transposon.

Short interspersed elements (SINEs) are parasites of APE LINEs. SINEs recruit the protein components of LINEs to integrate into the genome. As such SINEs represent, or at least approximate, the minimal RNA requirement for binding the LINE protein and for insertion into the genome. A SINE of a RLE LINE has been called a SIDE for Short Internally Deleted Elements. The RLE LINE R2 has SIDEs present in various Drosophila species that have the Hepatitis Delta Virus like ribozyme and the 3′ PBM RNA components of the parental LINE element (D. G. Eickbush, T. H. Eickbush, Mob DNA 3, 10 (2012)).

The ribozyme is used to cleave the element RNA from the rRNA/R2 cotranscript and is present in the parental R2 as well as the SIDE (Eickbush, et al., Mol Cell Biol (2010); Eickbush, et al., Mob DNA 3, 10 (2012)). Many of the HDV ribosozymes encoded by R2 elements cleave the rDNA/R2-element cotranscript such as to leave some ribosomal sequence at the 5′ end of the element RNA. As illustrated in the experiments presented below the target sequence, when present, is used to anneal to upstream target sequence post TPRT in order to form the 4-way junction integration-intermediate. The 4-way junction integration-intermediate is the gateway to the second half of the integration reaction. For R2 elements whose HDV trims off all target sequence, a template jump occurs to form the 4-way junction. The ribozyme may be optional in the engineered RNA because the RNA will not be made as a cotranscript. However, the presence of a ribozyme (e.g., HDV ribozyme) may help protect the element RNA from degradation by cellular RNAses. Additionally, the R2 protein may interact with the HDV ribozyme and/or aid in the integration reaction.

Presence of target sequence on the engineered RNA may aid in forming the 4-way junction particularly if using the protein and RNA components from an R2 element that is known to leave target sequence on its mRNA.

If CRISPR/Cas will be used to help drive the engineered RNA protein particle (RNP) either as a DNA binding domain or as a DNA binding plus DNA cleavage domain, then the RNA components of the an engineered CRISPR/Cas-9 system can be included in the engineered R2 “SIDE” RNA.

The 3′ PBM is an important RNA element. The 3′ PBM RNA is the only structural component of the RNA that binds to the R2 protein that is capable of undergoing TPRT, as such the 3′ PBM RNA would be an important component for the engineered RNA to be integrated into the genome. The sequence and structure of the 3′ PBM RNA used in the engineered RNA should be matched to the parental LINE RNA and the parental protein that binds to it.

The 5′ PBM RNA is not required for SIDE integration but is generally an important component of full-length integrated R2 elements. Its presence helps form an integration competent RNA protein particle (RNP), protect the RNA from degradation, and acts as a timing mechanism for entering the second half of the integration reaction (Christensen, et al., Proc Natl Acad Sci USA 103, 17602 (2006); see also the Examples below). Contained within the 5′ PBM is a suspected internal ribosome entry site (IRES) used by the R2 LINE to translate its mRNA. The IRES may have to be made non-functional (e.g., mutated, deleted, excluded, etc.) if the 5′ PBM RNA is used in the engineered RNA.

In the engineered RNA component, the LINE ORF sequence can be replaced with a gene or regulatory sequence of interest to be integrated into the genome.

2. Protein Component

The engineered RLE LINE protein is designed to bind to the RNA component and facilitate reverse transcription and integration of the gene of interest at the DNA target site alone or in combination with other endonucleases, reverse transcriptases, or accessory elements provided in trans. LINE based protein can include many or all of the protein domains of the open reading frame of a LINE transposon. Generally, the engineered LINE protein is designed to bind to the RNA component, bind to the genomic DNA, cleave the first strand of the target DNA, perform TPRT, bind to the 4-way junction intermediate, and cleave the 4-way junction and facilitate second strand synthesis.

The protein components are illustrated in FIG. 18B using a generic RLE ORF backbone as an example. The illustrated protein includes an N-terminal DNA binding domain (DB), RNA binding domain (RB), reverse transcriptase (RT), Linker including a presumptive α-Finger (αF) and a zinc-knuckle like CCHC motif, and the restriction-like DNA endonuclease (RLE).

The DB in R2Bm has a ZF and a myb. In R2Lp, R8Hm, and R9Av it has three ZFs and a myb. In NeSL-1 it has two ZFs. In R2Bm the myb is known to position a protein subunit downstream of the insertion site and to do so in the presence of 5′ PBM RNA (Christensen and Eickbush, Proc Natl Acad Sci USA 103, 17602 (2006)). In R2Lp, which targets the same site, the myb binds upstream of the target site. The sequence where the myb binds upstream of the insertion site is a degenerate palindrome of the downstream site (Thompson and Christensen, Mobile Genetic Elements 1, 29 (2011)). In NeSL the ZFs bind upstream of the insertion site and are believed aid in targeting the first strand cleavage (Shivram, et al., Mob Genet Elements 1, 169 (2011)). It is believed that the zinc finger in R2Bm, like in NeSL, is involved in targeting the first strand DNA cleavage (Shivram, et al., Mob Genet Elements 1, 169 (2011)). The R2 clade elements, which include R8 and R9, also use the ZFs and myb to aid in binding protein subunits to upstream and perhaps downstream sequences (Shivram, et al., Mob Genet Elements 1, 169 (2011)). As mentioned above, R2 SIDEs, lack the 5′ PBM RNA and as such do not pre-position a protein subunit downstream as does the parental LINE. The DB from the backbone LINE transposon can be mutated in place or substituted with a different DNA binding domain, for example, ZFs from a library or otherwise known ZF, or talens, or cas9, etc., in order to target a new site. The DB is believed to make contacts both upstream and downstream of the insertion site in the case of R2 elements, but only upstream target sequence in the case of NeSL-1. The engineered protein can be designed to bind to upstream sequences in some instances and to both upstream and downstream sequences in other instances.

The linker domain, as depicted in FIG. 18B, includes αF and a CCHC zinc knuckle-like domains (Mahbub, et al., Mob DNA 8, 16 (2017)). As illustrated in the experiments below, the αF and CCHC zinc knuckle position the target DNA for cleavage and synthesis at all stages of the integration reaction. The αF in particular is important for the binding and recognition of the 4-way junction. The 4-way junction is the gateway to second strand DNA cleavage and second strand DNA synthesis. In R2Bm the sequences downstream of the insertion site (i.e., the North arm of the 4-way junction) are important for DNA cleavage and are recognized by the DB. In the R2 LINE RNP a protein subunit is prebound to the downstream DNA sequences via association with the 5′ PBM RNA. The structure and sequence of the South, West, and East arms are also recognized by the protein. The R2 SIDE RNPs do not pre-position a protein subunit downstream of the insertion site, only at the upstream site. Elements like NeSL likely do not bind to sequences downstream of the insertion site via the DB. Instead, recognition of the 4-way junction and positioning of the endonuclease is done by the Linker, especially the αF. Recognition of the 4-way junction is both sequence specific and structure specific. The αF is thought to contact the heart of the 4-way junction similar to the αF of Prp8 binding to the multi-branched RNA at the 5′ splice site in the splicosome (Mahbub, et al., Mob DNA 8, 16 (2017)). See also the experiments below. Engineering of the RLE LINE protein to target new sites thus can include modification of the Linker, especially the αF, as well as the amino terminal DNA binding domain.

While much of the target cleavage specificity may come from the RLE being tethered to the DB and the Linker, the endonuclease does make some important contacts with the target DNA and appears to have some specificity (Govindaraju, et al., Nucleic Acids Res 44, 3276 (2016) and the experiments below). Thus, targeting the transposon to a new site can include modification of the RLE.

The RNA binding domain (RB) of R2Bm binds both 3′ and 5′ PBM RNAs (Jamburuthugoda and Eickbush, Nucleic Acids Res 42, 8405 (2014)). The RNA binding domain should be capable of binding the engineered transposon's RNA and in a manner that leads to reverse transcription and integration at the target site. Typically can be accomplished by using the parental protein and PBM RNAs from the same parental LINE. It may be advantageous, however, to use one parental LINE for the upstream 3′ PBM bound subunit, and another parental LINE for the downstream 5′ PBM bound subunit. The RNA binding domains can be mutated as needed to adjust for perturbations introduced by the engineering of the protein and RNA components.

FIGS. 18C and 18D illustrate two models of engineered transposon binding to the RNA component (18C) and reverse transcription and integration at the DNA target site (18D). The protein subunits are engineered to bind to the desired genomic location. Protein subunits can be from the same or from different parental RLE origin as different RLE lineages appear to use the amino-terminal DB in varying configurations for binding upstream and downstream of the insertion site. The design can also take into account the two insertion models (FIG. 18D): (1) a R2 LINE-like integration, and (2) a R2 SIDE-like integration.

Mutations (e.g., point mutations) in the DB, Linker, and the RLE will likely be needed in retargeting the element as DNA binding and recognition includes each of these domains.

B. Sources of Sequences for RNA and Protein Components

1. Parental Retrotransposons

The engineered retrotransposons are typically built from an existing LINE or SINE/SIDE, also referred to as a parental LINE or SINE/SIDE; or LINE or SINE/SIDE backbone. Thus appropriate nucleic acid sequences and amino acid sequences of LINEs and SINEs can be tailored, mutated or otherwise modified where needed to accomplish integration of the gene of interest at the target site of interest.

For example, RNA component sequences including, but not limited to, the 3′ PBM, which can be derived from a known RLE LINE or SIDE. The protein component sequences are typically derived from a RLE LINE. As discussed above, the RNA component and protein component should be compatible to ensure proper reverse transcription and integration of the gene of interest.

There are two major groups of LINEs. The two groups share a common RT and Linker (αF and IAP/gag-like CCHC zinc-knuckle). The two groups differ in their open reading frame (ORF) structures, RNA binding domains, DNA binding domains, and DNA endonuclease domains used to form the element RNP and to integrate into the host DNA.

The earlier branching group has a single ORF. The ORF encodes a multifunctional protein with N-terminal zinc finger and Myb motifs, an RT, a gag-knuckle like motif, and a type II restriction-like endonuclease (RLE) with a restriction endonuclease like fold (REL) (reviewed in Eickbush, et al., Microbiol Spectr. 2015; 3:MDNA3-0011. doi: 10.1128/microbiolspec.MDNA3-0011-2014; and Eickbush, “R2 and related site-specific non-long terminal repeat Retrotransposons.” In: Craig N L, Craigie R, Gellert M, Lambowitz A M, editors. Mobile DNA II. Washington, D.C.: ASM Press; 2002. p. 813-35.). This group of LINEs is generally site-specific during integration. The insect R2 element is a well-studied example of this early branching LINE group. Muhbub, et al., Mobile DNA (2017) 8:16 DOI 10.1186/s13100-017-0097-9n presents an updated model of the R2 RT along with an analysis of the linker region between the RT and the endonuclease. The R2 proteolytic data, in conjunction with sequence-structure alignments of the RT, linker, and RLE, indicate that RLE LINEs share a number of commonalities with the large fragment of Prp8, a highly conserved eukaryotic splicing factor that has a RT domain and an RLE domain.

RLE LINEs and their SIDEs can be used as the parental backbone and as a basis to derive the RNA and protein components of the engineered transposon.

2. Sources of DNA Binding Domains

In some embodiments, one or more DNA binding domains, or motifs therein, of a LINE or SINE can be modified or substituted with an alternative DNA binding domain. For example, N-terminal ZFs (and Myb motif if present) may represent the bulk of the targeting module for all site-specific RLE-bearing non-LTR retrotransposons that contain these motifs. The Myb and ZFs can undergo modification, allowing new sites to be targeted. During modification, individual ZF and Myb motifs can be acquired or lost. In addition, the physical/temporal linkage configurations between the various nucleic acid binding activities (5′ UTR RNA binding, 3′ UTR RNA binding, upstream DNA binding, and downstream DNA binding) and catalytic activities (first strand cleavage, TPRT, second strand cleavage, and second strand synthesis) may be reconfigured as elements transition to target new sites in the genome. Particular considerations related to integration and the linker region are also discussed above.

In some embodiments, the substitute DNA binding domain is derived from a DNA binding domain of a DNA binding protein or a motif thereof. Examples of DNA binding domains include, but are not limited to, helix-turn-helix, zinc finger, leucine zipper, winged helix, winged helix-turn-helix, helix-loop-helix, HMG-box, Wor3 domain, OB-fold domain, immunoglobulin fold, B3 domain, TAL effector, RNA-guided domain such as those in Cas proteins.

3. Sources of Transgenes

As introduced above the RNA component typically encodes a gene of interest, also referred to herein as a transgene, and an open reading frame of interest. In some embodiments the transgene sequence encodes one or more proteins or functional nucleic acids. The transgene can be monocistronic or polycistronic. In some embodiments, transgene is multigenic. As LINEs are in the 3-7 KB range and their SINEs/SIDEs a couple of hundred of bases, the transgene can be similarly sized. Larger transgenes may also be possible.

The disclosed engineered transposons can be used to induce gene correction, gene replacement, gene induction, gene tagging, transgene insertion, nucleotide deletion, gene disruption, gene mutation, etc. For example, the transposons can be used to add, i.e., insert or replace, nucleic acid material to a target DNA sequence (e.g., to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, the compositions can be used to modify DNA in a site-specific, i.e., “targeted”, way, for example gene knock-out, gene knock-in, gene editing, gene tagging, etc. as used in, for example, gene therapy, e.g., to treat a disease or as an antiviral, antipathogenic, or anticancer therapeutic

Thus, although the sequence of the RNA component to be integrated at the target site is typically referred to herein as a gene of interest, transgene, or an open reading frame of interest, it will be appreciated that in some embodiments the gene of interest is not a full-length gene or transgene, but rather a fragment of a gene, a regulatory element, or another untranslated element.

a. Polypeptide of Interest

The transgene(s) can encode one or more polypeptides of interest. The polypeptide can be any polypeptide. For example, the polypeptide of interest encoded by the transgene can be a polypeptide that provides a therapeutic or prophylactic effect to an organism or that can be used to diagnose a disease or disorder in an organism. The transgene can compensate for, or otherwise correct a genetic disease or disorder. The transgene can function in the treatment of cancer, autoimmune disorders, parasitic, viral, bacterial, fungal or other infections. The transgene(s) to be expressed may encode a polypeptide that functions as a ligand or receptor for cells of the immune system, or can function to stimulate or inhibit the immune system of an organism.

In some embodiments, the transgene(s) includes a selectable marker, for example, a selectable marker that is effective in a eukaryotic cell, such as a drug resistance selection marker. This selectable marker gene can encode a factor needed for the survival or growth of transformed host cells grown in a selective culture medium. Host cells not transformed with the selection gene will not survive in the culture medium. Typical selection genes encode proteins that confer resistance to antibiotics or other toxins, e.g., ampicillin, neomycin, methotrexate, kanamycin, gentamycin, Zeocin, or tetracycline, complement auxotrophic deficiencies, or supply important nutrients withheld from the media.

In some embodiments, the transgene(s) includes a reporter gene. Reporter genes are typically genes that are not present or expressed in the host cell. The reporter gene typically encodes a protein which provide for some phenotypic change or enzymatic property. Examples of such genes are provided in K. Weising et al. Ann. Rev. Genetics, 22, 421 (1988). Preferred reporter genes include glucuronidase (GUS) gene and GFP genes.

Additional genes including those that produce iPC, interleukins, receptors, transcription factors, and pro- and anti-apoptotic proteins.

b. Functional Nucleic Acids

The transgene(s) can encode a functional nucleic acid. Functional nucleic acids are nucleic acid molecules that have a specific function, such as binding a target molecule or catalyzing a specific reaction. Functional nucleic acid molecules can be divided into the following non-limiting categories: antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

c. Expression Elements

As introduced above, the transgene can include or be operably linked to expression control sequences that allow for transgene expression once integrated at the target DNA site. Operably linked means the disclosed sequences are incorporated into a genetic construct so that expression control sequences effectively control expression of a sequence of interest. Examples of expression control sequences include promoters, enhancers, and transcription terminating regions. A promoter is an expression control sequence composed of a region of a nucleic acid sequence molecule, typically within 100 nucleotides upstream of the point at which transcription starts (generally near the initiation site for RNA polymerase II).

Some promoters are “constitutive,” and direct transcription in the absence of regulatory influences. Some promoters are “tissue specific,” and initiate transcription exclusively or selectively in one or a few tissue types. Some promoters are “inducible,” and achieve gene transcription under the influence of an inducer. Induction can occur, e.g., as the result of a physiologic response, a response to outside signals, or as the result of artificial manipulation. Some promoters respond to the presence of tetracycline; “rtTA” is a reverse tetracycline controlled transactivator. Such promoters are well known to those of skill in the art. Commonly used promoter sequences and enhancer sequences are derived from Polyoma virus, Adenovirus 2, Simian Virus 40 (SV40), and human cytomegalovirus. DNA sequences derived from the SV40 viral genome may be used to provide other genetic elements for expression of a structural gene sequence in a mammalian host cell, e.g., SV40 origin, early and late promoter, enhancer, splice, and polyadenylation sites. Viral early and late promoters are particularly useful because both are easily obtained from a viral genome as a fragment which may also contain a viral origin of replication. Exemplary expression vectors for use in mammalian host cells are well known in the art.

To bring a coding sequence under the control of a promoter, it is preferable to position the translation initiation site of the translational reading frame of the polypeptide between one and about fifty nucleotides downstream of the promoter Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

C. Design Considerations

An important consideration in designing the engineered transposon is how the engineered transposon integrates into the target site. Modifications to the RNA and protein components should be carried out in manner that ensure integration of the gene of interest at the target site.

1. 4-way Branched DNA intermediate

Second-strand DNA cleavage has remained puzzling because the cleavage sites are generally not palindromic: The sequence around the second cleavage site is often unrelated to the sequence around the first strand site. In addition, the cleavages can produce blunt or staggered that lead to either a target site duplication or a target site deletion depending upon the stagger of the cleavage events for that element. The staggered cleavages can be a few bases away (e.g., 2 bp in R2Bm) or quite distant, e.g., 126 bp in R9 (Gladyshev and Arkhipova, Gene 448, 145 (2009), Christensen and Eickbush, J Mol Biol 336, 1035 (2004)). In APE LINEs, the cleavages are generally staggered such as to generate a modest 10-20 target site duplication upon insertion (Zingler, et al., Cytogenet Genome Res 110, 250 (2005); Christensen, et al. Genetica 110, 245 (2001); Ostertag, et al., Annu Rev Genet 35, 501 (2001)). The endonuclease from APE bearing LINEs (APE LINEs) appears to have some specificity for the first DNA cleavage site but much less so for the second on linear target DNA (Feng, et al., Cell 87, 905 (1996), Zingler, et al., Cytogenet Genome Res 110, 250 (2005), Christensen, et al. Genetica 110, 245 (2001), Feng, et al., Proc Natl Acad Sci USA 95, 2083 (1998), Maita, et al., Nucleic Acids Res 35, 3918 (2007)). The endonuclease from the RLE bearing LINEs (RLE LINEs) is similarly involved in target site recognition (Govindaraju, et al., Nucleic Acids Res 44, 3276 (2016)). In both cases, however, additional specifiers for cleavage have been invoked to account for the different specificity of the first and second strand cleavages including the endonuclease being tethered to the DNA by unidentified DNA binding domains in the protein. Another complicating factor is that the first cleavage event should occur in the presence of element RNA while the second cleavage event, according to a priori reasoning, should occur in the absence of element RNA, but this has been difficult to demonstrate in vitro (Christensen and Eickbush, Proc Natl Acad Sci USA 103, 17602 (2006)).

Second-strand DNA synthesis has remained unresolved for over 20 years and it has never been directly observed in vitro (Cost, et al., EMBO J 21, 5899 (2002), Zingler et al., Genome Res 15, 780 (2005), Han, Mob DNA 1, 15 (2010), Eickbush, et al., PLoS One 8, e66441 (2013), Kajikawa, et al., Gene 505, 345 (2012)). Second-strand synthesis is believed to be primed off of the free 3′-OH generated by the second-strand cleavage event and synthesized by the element encoded reverse transcriptase. It is unknown how the proposed primer-template association is generated as the target (ds)DNA ends drift away from each other post second strand DNA cleavage in in vitro reactions (Christensen and Eickbush, Mol Cell Biol 25, 6617 (2005), Christensen and Eickbush, Proc Natl Acad Sci USA 103, 17602 (2006)).

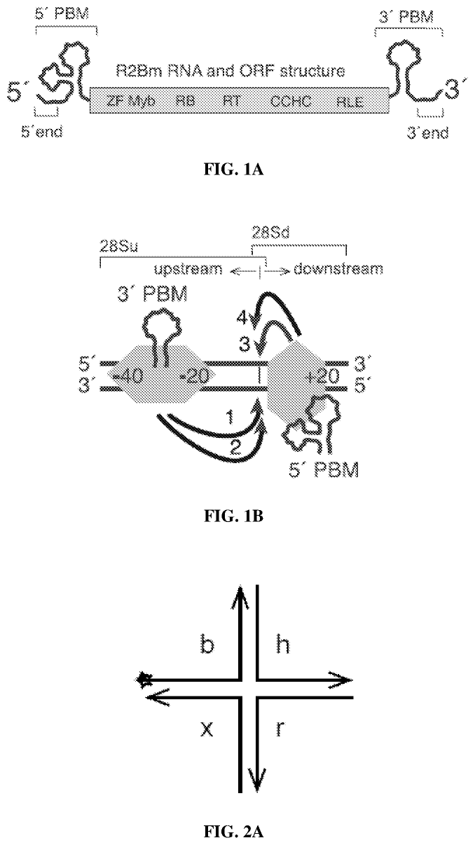

The R2 element from Bombyx mori, R2Bm, is one of a number of model systems that has been used to study the insertion reaction of LINEs (Eickbush and Eickbush, Microbiol Spectr 3, MDNA3 (2015)). R2 elements are site specific, targeting the “R2 site” in the 28S rRNA gene (Eickbush and Eickbush, Microbiol Spectr 3, MDNA3 (2015)). The R2 element encodes a single open reading frame with N-terminal zinc finger(s) (ZF) and myb domains (Myb), a central reverse transcriptase (RT), a restriction-like endonuclease (RLE), and a C-terminal gag-knuckle-like CCHC motif (FIG. 1A). The R2Bm protein has been expressed in E. coli and purified for use in in vitro reactions.

In vitro studies of the R2Bm protein and RNA have led to a model of integration for R2Bm (FIG. 1B) (Christensen and Eickbush, Proc Natl Acad Sci USA 103, 17602 (2006)). Two subunits of R2 protein, one bound to the 3′ protein binding motif (PBM) of the R2 RNA and other to the 5′ PBM, are thought to be involved in the integration reaction. The 5′ and 3′ PBM RNAs dictate the roles of the two subunits and coordinate a series of DNA cleavage and polymerization steps resulting in element integration by TPRT (FIG. 1A). The protein subunit bound to the element's 3′ PBM interacts with 28S rDNA sequences upstream of the R2 insertion site. The upstream subunit's RLE cleaves the first (bottom/antisense) DNA strand. After first-strand target-DNA cleavage, the subunit's RT performs TPRT using the 3′-OH generated by the cleavage event to prime first-strand cDNA synthesis. The protein subunit bound to the 5′ PBM RNA interacts with 28S rDNA sequences downstream of the R2 insertion site by way of the ZF and Myb domains. The downstream subunit's RLE cleaves the second (top/sense) DNA strand. Second-strand DNA cleavage, however, is not thought to occur until after the 5′ PBM RNA is pulled from the subunit, presumably by the process of TPRT, putting the protein in a “no RNA bound” conformation. Second-strand DNA cleavage does not occur in the absence of RNA in the in vitro reactions. Second strand cleavage had, until this report, needed a narrow range of R2 protein, 5′ PBM RNA, and target DNA ratios to be observed (Christensen and Eickbush, Proc Natl Acad Sci USA 103, 17602 (2006)). Additionally, second-strand cleavage divorced the upstream target-DNA from the downstream target-DNA making initiation of second-strand DNA synthesis from the upstream target-DNA to the TPRT product attached to the downstream target-DNA problematic (Christensen and Eickbush, Mol Cell Biol 25, 6617 (2005), Christensen and Eickbush, Proc Natl Acad Sci USA 103, 17602 (2006)).

The DNA endonuclease plays a central role in the integration reaction of LINEs. The RLE found in the early branching LINEs is a variant of the PD-(D/E)XK superfamily of endonucleases (Govindaraju, et al., Nucleic Acids Res 44, 3276 (2016), Yang, et al., Proc Natl Acad Sci USA 96, 7847 (1999)). LINE RLE have sequence and structural homology to archaeal Holliday junction resolvases (Govindaraju, et al., Nucleic Acids Res 44, 3276 (2016)). However, previous studies left open the question as to whether or not R2 protein could function as a Holliday junction resolvase and to what, if any, relevance this putative function might play in the insertion mechanism. The ability to of R2 protein to perform integration functions on branched DNAs was explored in the Examples below. The results indicate that an integration specific 4-way junction is an important intermediate and the gateway to the second half of the integration event. This 4-way junction is recognized by the RLE protein by both structure and sequence. The structure and sequence requirements can be used to facilitate the design of functional engineered transposons.

a. R2 Protein is not a General Holliday Junction Resolvase, but does Cleave its Own Integration Intermediate in a Resolvase-Like Reaction.

R2 protein was found to bind nonspecific 4-way DNA junctions, Holliday junctions, in preference to nonspecific linear DNA. The R2 protein appears to have a large surface for binding junction DNA when in the minus RNA conformation. This makes mechanistic sense in the context of R2 integration as it would be the minus RNA conformation of the R2 protein that would be likely to carry out second strand DNA cleavage. The presence of 5′ RNA abolished binding to the nonspecific junction DNA (and nonspecific DNA in general). It is not known what part of the R2 protein binds the 4-way DNA junction, it may not be the endonuclease. Indeed the experiments below implicate the Linker, especially the Linker's α-finger, as a major determinant of 4-way junction DNA recognition and binding. It is also unknown whether the 5′ PBM binding site overlaps the junction binding surface or if the lack of RNA promotes protein conformational changes that then reveal the junction binding surface. The binding surfaces for the 5′ and 3′ PBM RNAs are believed to be distributed across a large portion of the R2 protein, although currently the only identified RNA binding area is domain −1 and domain 0 (Jamburuthugoda and Eickbush, Nucleic Acids Res 42, 8405 (2014)). The CCHC zinc-knuckle has also been thought to bind to element RNA, but its true function has remained unknown. It could be that the 5′ PBM RNA forms a 4-way junction like mimic. The DNA binding surfaces of Holliday junction resolvases are large and highly positively charged, so it would make sense that R2 protein might make some use of this positive surface to bind help bind R2 RNA (Wyatt and West, Cold Spring Harb Perspect Biol 6, a023192 (2014)).

Although R2 binds to nonspecific DNA junctions in the absence of RNA, it was not able to subsequently resolve those junctions; DNA cleavages, particularly symmetrical DNA cleavages, did not occur. Therefore, R2 protein is not a Holliday junction resolvase in the strictest sense. However, with a more specific 4-way junction containing 28S rDNA and R2 sequences, the second/top-strand 28S rDNA cleavage event was nearly symmetrical with the bottom/first-strand cleavage that had been engineered into the 4-way junction. This DNA cleavage activity is very Holliday junction resolvase-like.

The presence of the template jump and the 5′ (South) arm being double stranded appeared to be the most important junction determinants, beyond the presence of target sequence in the downstream 28S rDNA (North) arm, for cleavability. A single stranded East arm is further stimulatory.

Interestingly, unless the R2 protein exists as a dimer in solution (of which there is no convincing evidence of), the bound versus DNA activity graph is linear and thus consistent with the endonuclease being monomeric (Christensen and Eickbush, Mol Cell Biol 25, 6617 (2005), Christensen and Eickbush, Proc Natl Acad Sci USA 103, 17602 (2006)). The DNA sequence at the center of the junction also might be important, but the constructs tested do not address this prospect as all of the R2 specific junctions contained 5-7 bases of 28S sequence to either side of the insertion site. In addition, each junction contained at least 25 bp of R2 5′ end sequence and 25 bp of R2 3′ end sequence. The R2 3′ arm appeared to be less important. Having the R2 3′ arm duplexed was even inhibitory. Removal of the R2 3′ arm, in an all DNA version was still cleavable, although only just. The presence of the first strand cleavage event appeared to also play a role in cleavability as a covalently closed all DNA version of the 4-way junction also had a difficult time being cleaved by R2 protein, although the lack of a RNA-DNA hybrids, especially in the 5′ arm, may have contributed to the reduced cleavability.

The presence of a full target site in the 4-way junction was inhibitory towards DNA cleavage unless the west arm (i.e., the 28S upstream DNA arm) included the template jump structure (“gap with a flap”). The data further indicate that the template-jump-derived West arm must be within a fairly narrow window of stability, too stable or rigid is inhibitory. Too low of a melting temperature leads to disassociation and/or formation of large of a single stranded flexible region and a concomitant loss of cleavage fidelity.

b. A New Model for R2Bm Integration

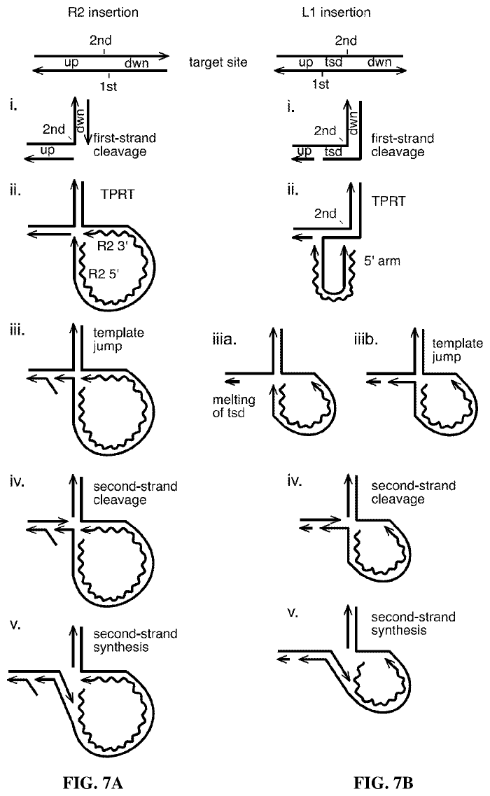

The deeper understanding of the second half of the insertion reaction for R2Bm has allowed for an improved R2Bm integration model to be put forth (FIG. 7A). The first half of the integration reaction is identical to steps 1 and 2 in FIG. 1B. After TPRT, however, the new model proposes a template-jump or recombination event from the 5′ end of the R2 RNA to the top-strand of the 28S rDNA upstream of the R2 insertion site forming a 4-way junction (step 3). It is this step that, to date, does not occur in vitro and may utilize host factors to form, if it exists at all. An association of the cDNA to the upstream target DNA is, however, consistent with a lot previous data and a 4-way junction presents a simple unified mechanism for 5′ junction formation, second strand DNA cleavage, and second strand DNA synthesis leading to full length element insertions.

The model makes sense of earlier in vivo experiments in which ‘upstream’ ribosomal RNA sequence attached to 5′ end of the R2Bm element RNA had been noted as a requirement for full length element insertion (Fujimoto et al., Nucleic Acids Res 32, 1555 (2004), Eickbush, et al., Mol Cell Biol 20, 213 (2000)). More recently, bioinformatic and in vitro studies of the R2 RNA transcript have determined that R2 RNA is co-transcribed with ribosomal RNAs as part of the same large transcript (Eickbush, et al., PLoS One 8, e66441 (2013), Eickbush and Eickbush, Mol Cell Biol (2010)). The R2 RNA is then processed from bulk of the ribosomal RNA by an HDV-like ribozyme found near the 5′ end of the R2 RNA (Eickbush, et al., PLoS One 8, e66441 (2013), Eickbush and Eickbush, Mol Cell Biol (2010)). For a number of R2 elements, however, the final processed R2 RNA retains some ribosomal RNA on the 5′ end, 27 nt of ribosomal RNA in the case of R2Bm (Eickbush, et al., PLoS One 8, e66441 (2013)). For elements that retain this much ribosomal RNA, the template jump may be more of a strand invasion or recombination event rather than a template jump (Fujimoto et al., Nucleic Acids Res 32, 1555 (2004); Eickbush, et al., Mol Cell Biol 20, 213 (2000)). For other R2 elements, however, the ribozyme leaves no ribosomal sequence on the processed R2 RNA (e.g., Drosophila simulans R2) and a template jump, as diagramed in FIG. 7A, is envisioned to occur (Kurzynska-Kokorniak, et al., J Mol Biol 374, 322 (2007), Eickbush, et al., PLoS One 8, e66441 (2013), Stage and Eickbush, Genome Biol 10, R49 (2009), Bibillo and Eickbush, J Mol Biol 316, 459 (2002)). The RT of both APE LINEs and RLE LINEs has been shown to have the ability to jump from the end one template to the beginning of another without any homology (Bibillo and Eickbush, J Mol Biol 316, 459 (2002)). Template jumps have long been believed to be involved in 5′ junction formation for both types of elements (Kurzynska-Kokorniak, et al., J Mol Biol 374, 322 (2007), Eickbush, et al., PLoS One 8, e66441 (2013), Stage and Eickbush, Genome Biol 10, R49 (2009), Bibillo and Eickbush, J Mol Biol 316, 459 (2002)). In addition to template jumping, LINE reverse transcriptases are able to use both DNA and RNA as a template during DNA synthesis and to displace a duplexed strand while polymerizing (Kurzynska-Kokorniak, et al., J Mol Biol 374, 322 (2007)).

Recently the R2 RLE's reported similarity to Archaeal Holliday junction resolvases, begged the question as to whether or not R2 can bind and cleave branched DNAs (Govindaraju, et al., Nucleic Acids Res 44, 3276 (2016), Mukha, et al., Front Genet 4, 63 (2013)). It turns out that the R2 protein can indeed bind to and cleave 4-way junctions in the absence of RNA. Second-strand DNA cleavage is step 4 in FIG. 7A. Second-strand cleavage occurs across from first-strand cleavage on R2 specific 4-way junctions, a reaction reminiscent of Holliday junction resolvase. Second-strand cleavage is dependent on both structure and sequence as sequences from the immediate insertion site area and downstream of the insertion site helped to drive cleavage.

The South arm, i.e., the R2 5′ arm, was an important cleavage determinant. The presence of 5′ PBM RNA prevents binding to non-specific 4-way junctions and prevents DNA cleavage of specific junctions. The R2 protein only cleaves in the absence RNA. The three way TPRT junction was not a good substrate for DNA cleavage.

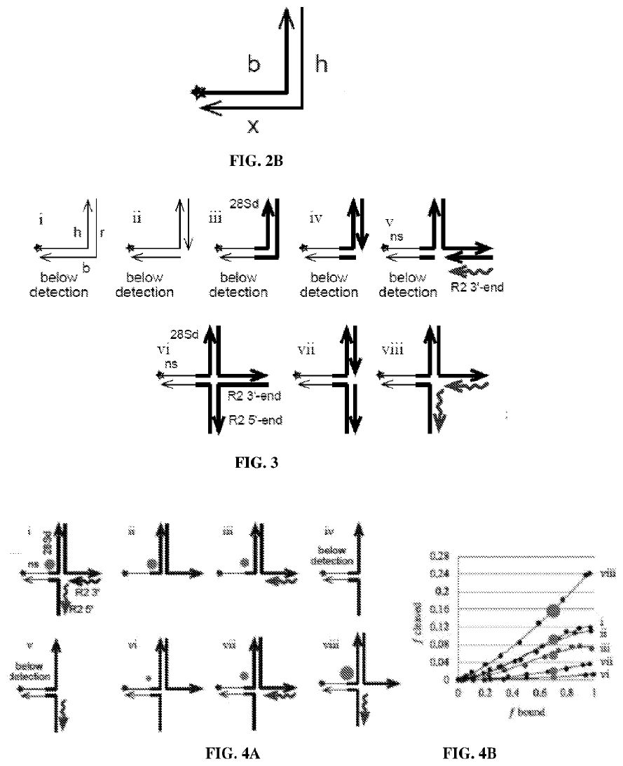

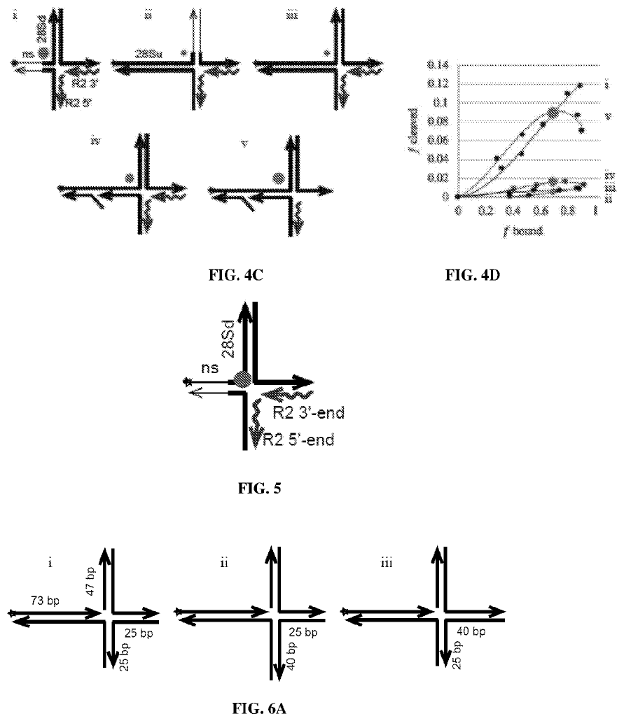

For elements with rRNA sequences at the 5′ end, like R2Bm, it is not clear what happens to the displaced RNA strand from the heteroduplex or the displaced ‘bottom strand’ target DNA flap while the cDNA strand is forming the junction depicted in FIG. 2-8A step 3, and what role, if any, the displaced strands plays in DNA cleavage. The displaced RNA was not included in the R2Bm integration 4-way junction constructs and the flap was non-specific DNA. In addition, it remains to be investigated as to whether or not the jump/recombination dislodges the upstream protein subunit as the 27 nt of ribosomal sequence encroaches on the minimal DNase footprint observed of the upstream subunit when the subunit is bound to linear 28S rDNA (Christensen and Eickbush, Mol Cell Biol 25, 6617 (2005), Christensen and Eickbush, J Mol Biol 336, 1035 (2004)). The construct in FIGS. 4A and 4C that contained the full target sequence along with a displaced target DNA strand behaved much more like the junctions lacking upstream target sequences than did junctions with full target sequence and no displaced target DNA. The recombined cDNA/target DNA duplex was 27 bp in these constructs matching that thought for R2Bm (Eickbush, et al., PLoS One 8, e66441 (2013)).

The fifth and final line of evidence in support of the model is that cleavage of the 4-way junction generates natural primer-template for second-strand DNA synthesis. The ‘downstream bound’ subunit appears prime second-strand DNA synthesis (FIG. 7A, step 5).

In vivo host factors may help keep junction halves held together long enough to prime second-strand synthesis. In vitro the primer template is released, at least when the upstream target DNA arm consists of nonspecific DNA.

c. Extrapolating the R2 Model to LINEs with Different Cleavage Staggers

The position of the second-strand DNA cleavage site relative to the first-strand cleavage site is quite variable across species even more so across the R2 clade. The stagger of the first and second DNA cleavage events in R2Bm is a small 5′ overhang of 2 bp that leads to 2 bp target site deletion upon insertion of the element. In Drosophila the R2 endonuclease produces blunt cleavages (Stage and Eickbush, Genome Biol 10, R49 (2009)). Other R2 elements produces small 3′ overhangs. The model presented in FIG. 7A works equally well for elements with any of these small staggers. The model can be adapted for elements with moderate 3′ overhang staggers by supposing a local melting or displacement of the TSD region followed by template switch to generate the 4-way junction. APE LINEs tend to produce a moderate 3′ overhanging stagger in the range of 10-20. It remains to be determined if APE LINEs use 4-way junction structure to drive second-strand DNA cleavage and synthesis. Bioinformatic analysis of 5′ junctions of full length L1 and Alu elements is indicative of template jumping to the upstream target sequence and that DNA repair process might be an alternative path to 5′ junction formation for abortive insertion events (Zingler et al., Genome Res 15, 780 (2005), Ichiyanagi, et al. N. Okada, Genome Res 17, 33 (2007), Gasior and Deininger, DNA Repair (Amst) 7, 983 (2008), Coufal, et al., Proc Natl Acad Sci USA 108, 20382 (2011), Richardson, et al., Microbiol Spectr 3, MDNA3 (2015)).

Twin priming in L1 might be a related, albeit aberrant, phenomenon to second-strand synthesis (Ostertag and Kazazian, Genome Res 11, 2059 (2001)). An association between the cDNA and the upstream target DNA has been believed for some R1 elements (Stage and Eickbush, Genome Biol 10, R49 (2009)). Ribosomal sequences are also important for element-RNA/target-DNA interactions during first strand synthesis for R1Bm as well as several other site-specific LINEs, but do not appear to be as important for R2Bm (Fujiwara, Microbiol Spectr 3, MDNA3 (2015), Anzai, et al., Nucleic Acids Res 33, 1993 (2005), Luan, et al., Mol Cell Biol 16, 4726 (1996)). A few LINEs have very larger staggers. The R9 Av element, an R2 clade member, produces a 126 bp stagger (Arkhipova, et al., Mob DNA 3, 19 (2012)). For large staggers, a D-loop opening allows for the template jump and formation of the 4-way junction.

d. Design Considerations for Maintaining Integration

In the design of the genomic DNA target site and the design of the engineered RNA that will be inserted into the genome by the engineered LINE protein care must be taken such that a productive 4-way junction will be formed during the integration reaction. The presence or absence of target sequence on the 5′ end of the engineered RNA will depend on whether or not the parental LINE's HDV leaves target sequence when it cleaves. Most of the ribozymes leave 10-25 nt of RNA derived from target DNA. The R2Bm ribozyme leaves target sequence. The R2Dm ribozyme does not. The target sequence remaining determines how the 4-way junction forms, how stable the West arm of the junction is, and the position and fidelity of the second-strand cleavage event. The West arm's stability (size of the template jump area) appears to be, in part, determined by how far upstream of the insertion site the upstream subunit is designed to bind. For R2 elements and NeSL this distance is about 10-20 bases upstream of the insertion site leaving room to form a West arm helix of about two turns. As R2BM is the parental LINE that most of the supporting biochemistry has been done on, R2Bm is a preferred parent LINE protein and parental RNA.

The stagger of the DNA cleavage event determines whether or not the East arm of the 4-way junction will be single or double stranded. A stagger that results in 3′ overhangs yields a 4-way junction with a single stranded East arm. A single stranded East arm is stimulatory for second strand DNA cleavage. In R2Bm the stagger is such that the East arm is a RNA/DNA duplex until such time as cellular RNAses remove the RNA from the East arm's RNA/DNA duplex.

As the South arm is also a major determinant for recognition and cleavage of the 4-way junction, the engineered RNA will need to maintain the sequence and structure elements of that arm by insuring that sequence at the 5′ end of the engineered that will become the South arm has the appropriate sequence and properties relative to the parental LINE protein/RNA.

2. Linker Region

LINEs integrate into new sites by a process called Target Primed Reverse Transcription (TPRT). The element encoded DNA endonuclease creates a nick in the host chromatin to expose a free 3′—OH group. The 3′-OH group is used by the element encoded reverse transcriptase to prime reverse transcription of the element RNA at the site of insertion. LINEs encode an invariant gag-like zinc-knuckle cysteine/histidine rich motif (CX2-3CX7-8HX4C) downstream of the reverse transcriptase (Jakubczak, et al., J. Mol. Biol. (1990). doi:10.1016/0022-2836(90)90303-4, Matsumoto, et al., Mol. Cell. Biol. 26, 5168-5179 (2006)). The spacing of the cysteines and histidine in the knuckle is unique to the knuckle found in LINEs. Immediately upstream of the zinc knuckle is a set of predicted helices (Mahbub, et al., Mob. DNA 8, 1-15 (2017)).

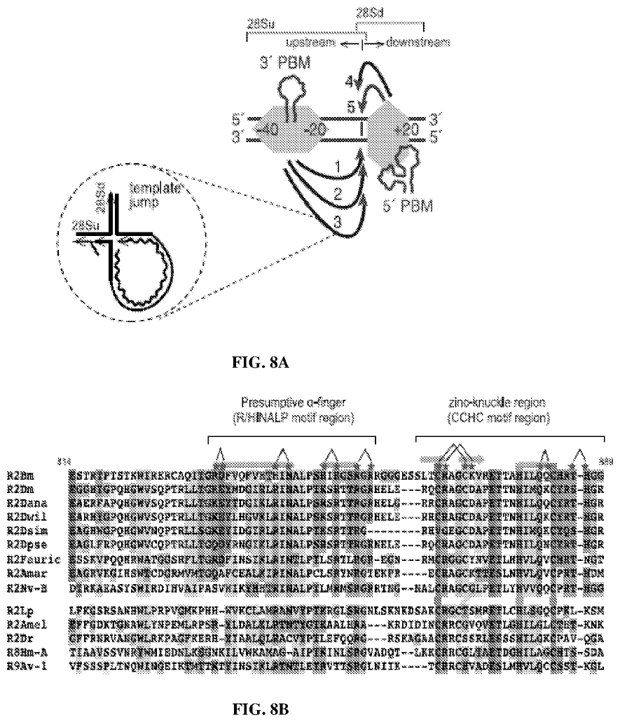

The R2 LINE from Bombyx mori (R2Bm) is a site specific LINE that has served as a model system in which to dissect the integration reaction of LINEs at the biochemical level as the protein can be purified in active form and used in in vitro assays (Jakubczak, et al., J. Mol. Biol. (1990). doi:10.1016/0022-2836(90)90303-4, Kojima, et al., Mol. Biol. Evol. (2006). doi:10.1093/molbev/ms1067; Gladyshev, et al., Gene (2009). doi:10.1016/j.gene.2009.08.016). The R2 ORF encodes a multifunctional protein with N-terminal zinc-finger(s) (ZF) and myb domains that are involved in DNA binding; an RNA binding (RB) domain; a central reverse transcriptase (RT); a linker region containing several conserved predicted helices (HINALP motif), and a gag-like zinc knuckle (CCHC motif), and a PD-(D/E)XK type II restriction-like endonuclease (RLE) domain (FIG. 1A) (Jakubczak, et al., J. Mol. Biol. (1990). doi:10.1016/0022-2836(90)90303-4, Mahbub, et al., Mob. DNA 8, 1-15 (2017), Burke, et al., Mol. Cell. Biol. (1987). doi:10.1128/MCB.7.6.2221.Updated, Yang, et al., Proc. Natl. Acad. Sci. U.S.A 96, 7847-52 (1999), Christensen, et al., Nucleic Acids Res. 33, 6461-6468 (2005), Jamburuthugoda, et al., Nucleic Acids Res. 42, 8405-8415 (2014), Christensen, et al., Mol. Cell. Biol. 25, 6617-6628 (2005)) The R2 RNA sequence corresponding to the 5′ and 3′ untranslated region (UTR) folds into distinct structures that are known to bind R2 protein, and hence are termed as 5′ PBM and 3′ PBM, respectively (FIG. 1A) (Kierzek, et al., Nucleic Acids Res. (2008), doi:10.1093/nar/gkm1085, Kierzek, et al., J. Mol. Biol. 390, 428-442 (2009), Christensen, et al., Proc. Natl. Acad. Sci. U.S.A 103, 17602-17607 (2006)). Binding to the 5′ PBM and 3′ PBM RNAs control protein conformation and role in the integration reaction (FIG. 8B) (Christensen, et al., Mol. Cell. Biol. 25, 6617-6628 (2005)). Selective addition of the RNA, DNA, and protein components allow for distinct stages of the integration reaction to be assayed.

R2 protein bound to 3′ PBM adopts a conformation that allows the protein to bind the upstream 28S DNA sequences (28Su) relative to the insertion site. The domain(s) of the R2 protein that contacts the 28Su to form upstream protein subunit remain largely unidentified (Govindaraju, et al., Nucleic Acids Res. 44, 3276-3287 (2016), Thompson, et al., Elements 1, 29-37 (2011), Shivram, et al., Mob. Genet. Elements 1, 169-178 (2011).). R2 protein bound to the 5′ PBM adopts a conformation that allows the protein to bind the downstream 28S DNA sequences (28Sd). The ZF and Myb motifs of R2 protein include major residues that are known to interact with the 28Sd forming downstream protein subunit (Christensen, et al., Nucleic Acids Res. 33, 6461-6468 (2005)). The upstream and downstream protein subunits catalyze the integration of R2 elements in two half reactions each including DNA cleavage followed by DNA synthesis (Christensen, et al., Mol. Cell. Biol. 25, 6617-6628 (2005)). The five steps of integration are: (1) The endonuclease from upstream subunit nicks the target DNA exposing a 3′-OH at the insertion site; (2) The exposed 3′-OH is used as a primer by the upstream subunit's reverse transcriptase for TPRT; (3) A template jump or recombination event occurs where the cDNA from the 5′ end of the reverse transcribed becomes associated with the upstream target DNA sequences to form a four-way junction; (4) The downstream subunit cleaves the four-way DNA junction; (5) the 3′-OH generated by the cleavage event is used as the primer for second strand DNA synthesis of the element.

The role of the linker region, located after the RT in all LINEs, has previously remained illusive (Mahbub, et al., Mob. DNA 8, 1-15 (2017)). Point mutations were introduced into the linker's gag-like zinc knuckle and presumptive α-finger (FIG. 8B). The spacing of the CCHC motif is unique to LINEs (Malik, et al., Mol. Biol. Evol. 16, 793-805 (1999), Fanning and Singer, Nucleic Acids Res. (1987). doi:10.1093/nar/15.5.2251). In a previous in vivo study using APE bearing human LINE-1 elements, mutating the first two cysteines in the linker region's CCHC motif significantly reduced LINE-1 retrotransposition (Moran, et al., Cell 87, 917-927 (1996)). In another in vivo study with human LINE-1, reduced levels of RNP complex was observed when first two cysteines were mutated which indicated its possible role in nucleic acid binding (Doucet, et al., PLoS Genet. 6, 1-19 (2010)). When the zinc knuckle structure was altered by substituting first three cysteines into serine, no reduction in RNA binding activity was reported for human LINE-1 elements in vitro (Piskareva, et al., FEBS Open Bio 3, 433-437 (2013)). However, in the same study, sequences C-terminal to the RT was found to be involved in RNA binding. Mutating residues upstream of the presumptive α-finger in LINE-1 elements reduced retrotransposition activity in in vivo (Moran, et al., Cell 87, 917-927 (1996)). The helices upstream of the zinc knuckle, along with the zinc knuckle itself, reportedly align with the α-finger and the non-zinc knuckle of the eukaryotic splicing factor, Prp8 (Mahbub, et al., Mob. DNA 8, 1-15 (2017), Wan, et al., Science (80-.). (2016). doi:10.1126/science.aad6466, Bertram, et al., Cell (2017). doi:10.1016/j.cell.2017.07.011).

The Examples below test the effect of a series of double mutations generated throughout the presumptive α-finger and zinc knuckle of R2Bm on in vitro function under conditions that test for DNA binding, first-strand DNA cleavage, first-strand DNA synthesis, second-strand DNA cleavage, and second-strand DNA synthesis. The results lead to conclusions that can be used to facilitate the design of functional engineered transposons.

a. The Primary Role of the Linker does not Appear to be Binding Element RNA.

The CCHC mutations reduced the accumulation of ORF2 protein into ribonucleoprotein (RNP) complex, implying a possible role in binding element RNA (Doucet, et al., PLoS Genet. 6, 1-19 (2010)). Likewise, sequences upstream of the presumptive α-finger were found to reduce retrotransposition activity in vivo (Moran, et al., Cell 87, 917-927 (1996)). Domain swapping experiments between the human and mouse L1 elements also indicate that sequence just upstream of the zinc knuckle are important for retrotransposition in vivo (Wagstaff, et al., PLoS One 6, (2011)). The upstream sequences are functionally linked to the zinc knuckle and other parts of the protein in a complicated yet modular way that is not well understood. A number of these domain swaps were in the middle of the presumptive α-finger. In addition, a polypeptide containing 180 amino acids of the C-terminal end of ORF2 of L1Hs containing much of the α-finger and the zinc knuckle was found to bind non-specifically to RNA in vitro, but mutating the cysteines did not affect nucleic acid binding (Piskareva, et al., FEBS Open Bio 3, 433-437 (2013)).

The in vitro study has found that mutations in the zinc knuckle and α-finger in R2Bm do not overtly reduce binding to the element 5′ PBM RNA or to 3′ PBM RNA. It should be noted, however, that RNA binding is inferred by the formation of distinct DNA-RNA-protein complexes in the EMSA gels (Jamburuthugoda, et al., Nucleic Acids Res. 42, 8405-8415 (2014), Christensen, et al., Proc. Natl. Acad. Sci. U.S.A 103, 17602-17607 (2006)). Protein-DNA and Protein-DNA-RNA complexes with either the 5′ PBM RNA or 3′ PBM RNA have unique well defined migration patterns in EMSA gels (Christensen, et al., Mol. Cell. Biol. 25, 6617-6628 (2005)). Amino acids that affect incorporation of the RNA into the protein-nucleic acid complexes can thus be detected as a change in the ratio of Protein-DNA to Protein-DNA-RNA complexes in the generic protein titration series. The RT −1 and RT 0 domains were determined to be RNA binding domains using an identical assay system (Jamburuthugoda, et al., Nucleic Acids Res. 42, 8405-8415 (2014)). RNA titrations instead of protein titrations were also carried out on several of the mutants with no indication of changes to RNA binding. That said, an RNA binding role cannot be ruled out. The RNA binding surface might be too large and widely distributed across the surface of the R2 protein for point mutants to make an observable difference in the assays. This is one reason why double point mutants were used, instead of single point mutants (Jamburuthugoda, et al., Nucleic Acids Res. 42, 8405-8415 (2014)).

Mutations to the core CCHC motif of the zinc knuckle (C/SC/SHC) and to the HINALP motif of the presumptive α-finger (H/AIN/AALP) are consistent with local disruption of protein structure leading an inability to form stable gel migrating protein-nucleic acid complexes in EMSA gels. It was undiscernible from the EMSA with these two mutants if RNA was bound or not as no distinct Protein-DNA or Protein-DNA-RNA bands were observed. All other mutations in the zinc knuckle and α-finger regions retained the ability to efficiently form the proper protein-RNA-DNA complexes in patterns similar to WT protein.

b. The Linker Presents Nucleic Acids to the RLE and RT During the First Half of the Integration Reaction.

A comparative summary of the DNA binding, cleavage, and synthesis results for each of the mutants tested in this study is presented in Table 2 below. Mutations to the core of the CCHC motif (C/SC/SHC) and to the core of the HINALP motif (H/AIN/AALP) lead to an unrestrained DNA endonuclease and an inability to form stable upstream bound protein-nucleic acid complexes. All other mutants are able to form normal upstream protein-RNA-DNA complexes. Two of the α-finger mutations (SR/AIR/A and SR/AGR/A) led to the endonuclease being overly restrained and not cleaving. The inability to perform first strand cleavage was not related to the mutant's ability to bind to upstream DNA sequences as one of the mutants was unimpaired in DNA binding in the presence of 3′ PBM RNA and the other mutation actually increased the protein's ability to bind to target DNA in the presence of 3′ PBM RNA. Rather, resides R849, R851, R854, and R856 are used to position the target DNA and/or the DNA endonuclease for first-strand DNA cleavage.

Once cleaved, α-finger GR/AD/A and SR/AIR/A mutants were unable to perform first strand cDNA synthesis (TPRT) on pre-nicked target DNA indicating a role of the mutated residues in positioning the RT and/or nucleic acid components relative to each other. Indeed, the GR/AD/A mutant lacked any other major phenotype beyond the inability to perform TPRT and a modest reduction in binding to upstream DNA sequences. The zinc knuckle mutants CR/AAGCK/A, HILQ/AQ/A, and RT/AH/A modestly reduced first strand DNA cleavage and retained near wild type first-strand DNA synthesis activity. Upstream DNA binding was not carefully examined, but appeared to be normal.

c. The Linker Region is Key to the Second Half of the Integration Reaction.

The second half of the integration reaction begins with R2 protein being associated with the 5′ PBM RNA and thus becoming bound to DNA sequences downstream of the insertion site on linear target DNA. Mutations to the core of the CCHC motif (C/SC/SHC) and to the core of the HINALP motif (H/AIN/AALP) lead to an unrestrained DNA endonuclease and an inability to form stable downstream bound protein-nucleic acid complexes. All other mutants were able to form normal downstream protein-RNA-DNA complexes on linear target DNA and appeared to have minimal effect on binding to linear DNA. That said, the SR/AIR/A mutation did show a modest decrease in binding to the downstream sequence on linear DNA and the zinc knuckle mutants were not quantitatively tested.

The second half of the integration only proceeds when the downstream subunit is in the “no-RNA-bound” state (Christensen, et al., Proc. Natl. Acad. Sci. U.S.A 103, 17602-17607 (2006)). Although second-strand DNA cleavage can occur on linear DNA, it needs a complicated set of 5′ RNA, DNA, and protein ratios to do so and is non-productive in the sense that second-strand synthesis does not occur (Christensen, et al., Mol. Cell. Biol. 25, 6617-6628 (2005), Christensen, et al., Proc. Natl. Acad. Sci. U.S.A. 103, 17602-17607 (2006)). For this reason, it is now thought that the second half of the integration reaction, specifically second-strand DNA cleavage and second-strand synthesis, mechanistically needs the formation of the 4-way junction (see Example 1-8). The 4-way junction appropriately cleaves the junction in the absence of RNA and the cleaved product is a substrate for second strand synthesis (see Example 1-8).

All of the zinc knuckle and α-finger mutants tested, except for the CR/AAGCK/A mutant, were unable to perform second-strand cleavage on linear DNA (Table 2), yet, importantly, the zinc knuckle mutants did not impair second-strand cleavage on the more important 4-way junction. The α-finger mutations that lie closest to the zinc knuckle, SR/AIR/A and SR/AGR/A, greatly reduce binding to the 4-way junction and abolish second-strand DNA cleavage. Second-strand synthesis was similarly affected by the two sets of mutations. The results indicate that the α-finger is important for 4-way junction recognition as well as presenting the bound DNA to the endonuclease and to the reverse transcriptase. The zinc knuckle mutants HILQ/AQ/A and RT/AH/A severely reduced second-strand synthesis indicating that the zinc knuckle residues are involved in positioning the cleaved junction and/or the reverse transcriptase for primer extension.

d. Structural and Functional Connections to APE LINEs and to Prp8

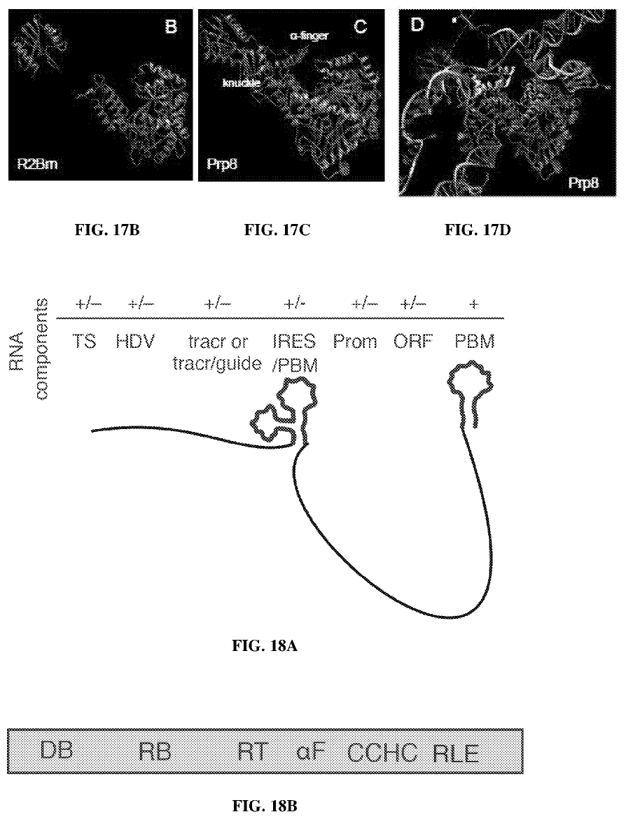

The protein encoded by R2Bm has been determined to consist of two globular domains. The larger of the two domains (colored in FIG. 17A-17D) contains the RT, the RLE, and a region between the two called the linker (Mahbub, et al., Mob. DNA 8, 1-15 (2017)). The end of the linker region contains an invariant zinc knuckle and several conserved helices upstream of the zinc knuckle. The upstream helices are referred to here as the “presumptive α-finger” of which the HINALP motif is central to the α-finger in R2Bm. APE LINEs also contain a “linker” with a presumptive α-finger and a zinc knuckle located beyond the RT (FIG. 17A-17D).

The large globular domain of R2Bm, an RLE LINE, shares structural as well as sequence similarities to the large fragment of eukaryotic splicing factor Prp8 (see FIG. 17A-17D). Prp8 has an RT, an RLE, and a linker region between the RT and RLE. Towards the end of the linker region in Prp8 is a non-zinc knuckle structure. Upstream of the non-zinc knuckle are a set of helices that align with the helices found upstream of the zinc knuckle in LINEs. The helices upstream of the non-zinc knuckle in Prp8 form a very prominent and important α-finger. The α-finger protrudes out over the reverse transcriptase (see FIG. 17C) (Bertram, et al., Cell (2017). doi:10.1016/j.cell.2017.07.011). It is by analogy to the α-finger in Prp8 that the corresponding region of the RLE LINEs is called the “presumptive α-finger” (Mahbub, et al., Mob. DNA 8, 1-15 (2017)). In Prp8 the non-zinc knuckle, the α-finger, and the RT thumb work together to bind the splice sites and spliceosomal RNAs. The non-zinc knuckle and the α-finger are dynamic in Prp8 undergoing/promoting protein and protein-RNA conformational changes across all aspects of the splicing reaction. Of particular interest is the fact that in the U4/U6.U5 tri-snRNP and in the B complex the α-finger and non-zinc knuckle bind to important branched RNA structures.

The data reported here indicates that whatever the actual structure of the R2Bm linker is, the linker is central to the recognition of the 4-way junction integration intermediate. It also acts as a protein-DNA conformational switch or hub for correctly positioning the EN, the RT, and the substrate DNA relative to each other.

e. Design Considerations for Maintaining Integration

The linker region is an important DNA binding region and protein-nucleic acid conformational control region. The linker region makes specific and non-specific contacts. Both the α-finger and the IAP/Gag-like zinc knuckle modulate the DNA cleavage and DNA synthesis events. The α-finger in particular plays a role in binding to the 4-way junction. It is thought that the α-finger contacts the center of the 4-way junction, like the α-finger in Prp8 which sits at the center of the 5′ splice site, a multibranched RNA structure. It is likely that transposon α-finger also makes base specific contacts in addition to nonspecific contacts. The Linker is also thought to be involved in binding to the LINE RNA. In designing the engineered LINE protein, the engineered RNA, and the target DNA, care must be taken so as to either maintain the parental protein contacts between certain target DNA sequences and RNA sequences or mutate the Linker such that it will make newly desired DNA/RNA contacts.

### III. Methods of Use

The disclosed compositions can be used to ex vivo or in vivo for introduce of genes of interest at DNA targets sites of interest. For example, in preferred embodiments the RNA component and protein component of the engineered transposon are delivered to, or otherwise expressed in a cell and the gene of interest is integrated into the genome of the cell at a DNA target site of interest. The RNA component can be delivered as RNA, or as DNA encoding the RNA component (e.g., an expression vector). The protein component can be delivered as protein, or as RNA or DNA encoding the protein component (e.g., an expression vector). In some embodiment, vectors encoding the protein are expressed in bacterial or eukaryotic expression system, and the protein harvested and delivered to the target cells. In some embodiment, RNA is prepared by in vitro transcription, and/or protein is prepared by in vitro transcription/translation. The RNA and protein components can be expressed from the same or different vectors.

A. Vectors and Host Cells

Vectors and host cells for preparing engineered transposons are also provided. Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, Conn.), maltose E binding protein and protein A.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection.

Useful prokaryotic and eukaryotic systems for expressing and producing polypeptides are well known in the art include, for example, Escherichia coli strains such as BL-21, and cultured mammalian cells such as CHO cells.

B. Methods of Editing Cellular Genomes

The methods typically include contacting a cell with an effective amount of engineered transposon to modify the cell's genome. As discussed herein contacting cells with an engineered retrotransposon means that both an RNA component and a protein component are present in that same cell(s) at the same time. In some embodiments, the RNA and protein components are mixed together before contact with the cell. In some embodiments, they are contacted with the cell separately and form a complex for the first time within the cell. In some embodiments, one or both components are delivered as DNA expressed in the cell. Any of the embodiments can include use of electroporation, lipofection, calcium phosphate, or calcium chloride co-precipitation, DEAE dextran, or other suitable transfection methods to facilitate delivery of nucleic acids or protein to the cells.

As discussed in more detail below, the contacting can occur ex vivo or in vivo. In preferred embodiments, the method includes contacting a population of target cells with an effective amount of engineered retrotransposon achieve a therapeutic result.

For example, the effective amount or therapeutically effective amount can be a dosage sufficient to treat, inhibit, or alleviate one or more symptoms of a disease or disorder, or to otherwise provide a desired physiologic effect, for example, reducing, inhibiting, or reversing one or more of the underlying pathophysiological mechanisms underlying a disease or disorder.

The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids and proteins. The precise dosage will vary according to a variety of factors such as subject-dependent variables (e.g., age, immune system health, clinical symptoms etc.).

1. Ex Vivo Gene Therapy

In some embodiments, ex vivo gene therapy of cells is used for the treatment of a disease or disorder, including but not limited to, genetic disorders in a subject. For ex vivo gene therapy, cells can be isolated from a subject and contacted ex vivo with the compositions to produce cells containing the inserted transgene. In a preferred embodiment, the cells are isolated from the subject to be treated or from a syngenic host. Target cells are removed from a subject prior to contacting with an engineered retrotransposon. In some embodiments, the cells are hematopoietic progenitor or stem cells. In a preferred embodiment, the target cells are CD34⁺ hematopoietic stem cells. Hematopoietic stem cells (HSCs), such as CD34+ cells are multipotent stem cells that give rise to all the blood cell types including erythrocytes. Therefore, CD34+ cells can be isolated from a patient with, for example, thalassemia, sickle cell disease, or a lysosomal storage disease, the mutant gene altered or repaired ex-vivo using the disclosed compositions and methods, and the cells reintroduced back into the patient as a treatment or a cure.

Stem cells can be isolated and enriched by one of skill in the art. Methods for such isolation and enrichment of CD34+ and other cells are known in the art and disclosed for example in U.S. Pat. Nos. 4,965,204; 4,714,680; 5,061,620; 5,643,741; 5,677,136; 5,716,827; 5,750,397 and 5,759,793. As used herein in the context of compositions enriched in hematopoietic progenitor and stem cells, “enriched” indicates a proportion of a desirable element (e.g. hematopoietic progenitor and stem cells) which is higher than that found in the natural source of the cells. A composition of cells may be enriched over a natural source of the cells by at least one order of magnitude, preferably two or three orders, and more preferably 10, 100, 200 or 1000 orders of magnitude.

Once progenitor or stem cells have been isolated, they may be propagated by growing in any suitable medium. For example, progenitor or stem cells can be grown in conditioned medium from stromal cells, such as those that can be obtained from bone marrow or liver associated with the secretion of factors, or in medium including cell surface factors supporting the proliferation of stem cells. Stromal cells may be freed of hematopoietic cells employing appropriate monoclonal antibodies for removal of the undesired cells.

The modified cells can also be maintained or expanded in culture prior to administration to a subject. Culture conditions are generally known in the art depending on the cell type.

In other embodiments, the technology is used as part of CAR T-based therapy. Immune cells are harvested (e.g., T cells) are taken from a patient's blood. A chimeric antigen receptor (CAR) introduced into a target site in the cells' genome using an engineered transposon. Large numbers of the CAR T cells can be grown in the laboratory and given to the patient by infusion. CAR T-cell therapy is used in the treatment of some types of cancer.

2. In vivo Gene Therapy

The disclosed compositions can be administered directly to a subject for in vivo gene therapy.

a. Pharmaceutical Formulations

The disclosed compositions are preferably employed for therapeutic uses in combination with a suitable pharmaceutical carrier. Such compositions include an effective amount of the composition, and a pharmaceutically acceptable carrier or excipient.

It is understood by one of ordinary skill in the art that nucleotides administered in vivo are taken up and distributed to cells and tissues (Huang, et al., FEBS Lett., 558(1-3):69-73 (2004)). For example, Nyce, et al. have shown that antisense oligodeoxynucleotides (ODNs) when inhaled bind to endogenous surfactant (a lipid produced by lung cells) and are taken up by lung cells without a need for additional carrier lipids (Nyce, et al., Nature, 385:721-725 (1997)). Small nucleic acids are readily taken up into T24 bladder carcinoma tissue culture cells (Ma, et al., Antisense Nucleic Acid Drug Dev., 8:415-426 (1998)).

The disclosed compositions may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles, nanoparticles, or microspheres formed of biodegradable or non-biodegradable polymers or proteins or liposomes for targeting to cells. Such systems are well known to those skilled in the art and may be optimized for use with the appropriate nucleic acid.

Various methods for nucleic acid delivery are described, for example, in Sambrook et al., Molecular Cloning: A Laboratory Manual, Cold Spring Harbor Laboratory, New York (1989); and Ausubel, et al., Current Protocols in Molecular Biology, John Wiley & Sons, New York (1994). Such nucleic acid delivery systems include the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. The nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, optionally with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents and inert gases. In addition, sterile, fixed oils are conventionally employed as a solvent or suspending medium. For this purpose any bland fixed oil including synthetic mono- or di-glycerides may be employed. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The disclosed compositions alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, the compositions include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, nucleic acids are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett., 14(19):4975-4977 (2004)) and in vivo (Soutschek, et al., Nature, 432(7014):173-178 (2004)). In addition, it has been shown that binding of steroid conjugated oligonucleotides to different lipoproteins in the bloodstream, such as LDL, protect integrity and facilitate biodistribution (Rump, et al., Biochem. Pharmacol., 59(11):1407-1416 (2000)). Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines U.S. Pat. No. 6,919,208 to Levy, et al., also describes methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

b. Methods of Administration

In general, methods of administering nucleic acid and protein compositions are well known in the art. In particular, the routes of administration already in use for nucleic acid therapeutics, along with formulations in current use, provide preferred routes of administration and formulation for the engineered transposons described above. Preferably the compositions are injected into the organism undergoing genetic manipulation, such as an animal requiring gene therapy.

The disclosed compositions can be administered by a number of routes including, but not limited to, oral, intravenous, intraperitoneal, intramuscular, transdermal, subcutaneous, topical, sublingual, rectal, intranasal, pulmonary, and other suitable means. The compositions can also be administered via liposomes. Such administration routes and appropriate formulations are generally known to those of skill in the art.

Administration of the formulations may be accomplished by any acceptable method which allows the gene editing compositions to reach their targets.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The compositions may be delivered in a manner which enables tissue-specific uptake of the agent and/or nucleotide delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the composition, over a certain time period, for example, hours, days, weeks, months or years. This may be accomplished, for example, by repeated administrations of a formulation or by a sustained or controlled release delivery system in which the compositions are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the oligonucleotides are contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the engineered transposon. In addition, a pump-based hardware delivery system may be used to deliver one or more embodiments.

Examples of systems in which release occurs in bursts include systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

c. Preferred Formulations for Mucosal and Pulmonary Administration

Active agent(s) and compositions thereof can be formulated for pulmonary or mucosal administration. The administration can include delivery of the composition to the lungs, nasal, oral (sublingual, buccal), vaginal, or rectal mucosa.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

C. Diseases to be Treated

The disclosed engineered transposons are especially useful to treat genetic deficiencies, disorders and diseases caused by mutations in single genes, for example, to correct genetic deficiencies, disorders and diseases caused by point mutations. If the target gene contains a mutation that is the cause of a genetic disorder, then the disclosed compositions can be used for mutagenic repair that may restore the DNA sequence of the target gene to normal. The target sequence can be within the coding DNA sequence of the gene or within an intron. The target sequence can also be within DNA sequences that regulate expression of the target gene, including promoter or enhancer sequences. The disclosed transposons can additionally or alternatively deliver a wildtype or even and enhance version of the gene of interest, or deliver new (e.g., heterologous) gene to the cell. Thus, the technology can repair or replace genes, supplement genes, or add new genes.

If the target gene is an oncogene causing unregulated proliferation, such as in a cancer cell, then the engineered transposon is useful for causing a mutation that inactivates the gene and terminates or reduces the uncontrolled proliferation of the cell. The engineered transposon is also a useful anti-cancer agent for activating a repressor gene that has lost its ability to repress proliferation. The target gene can also be a gene that encodes an immune regulatory factor, such as PD-1, in order to enhance the host's immune response to a cancer. Thus, the engineered transposon can be designed to reduce or prevent expression of PD-1, and administered in an effective amount to do so.

The engineered transposon can be used as antiviral agents, for example, when designed to modify a specific a portion of a viral genome needed for proper proliferation or function of the virus.

### EXAMPLES

Muhbub, et al., Mobile DNA (2017) 8:16 DOI 10.1186/s13100-017-0097-9, is specifically incorporated by reference herein in its entirety.

### Example 1: R2 Protein Binds Preferentially to a Nonspecific 4-Way Junction DNA Over Nonspecific Linear DNA

### Materials and Methods

Protein Purification

R2Bm protein expression and purification were carried out as previously published (Govindaraju, et al., Nucleic Acids Res 44, 3276 (2016)). Briefly, BL21 cells containing the R2 expression plasmid were grown in LB broth and induced with IPTG. The induced cells were pelleted by centrifugation, resuspended, and gently lysed in a HEPES buffer containing lysozyme and triton X-100. The cellular DNA and debris were spun down and the supernatant containing the R2Bm protein was purified over Talon resin (Clontech #635501). The R2Bm protein was eluted from the Talon resin column and stored in protein storage buffer containing 50 mM HEPES pH 7.5, 100 mM NaCl, 50% glycerol, 0.1% triton X-100, 0.1 mg/ml bovine serum albumin (BSA), and 2 mM dithiothreitol (DTT) and stored at −20° C. R2 protein was quantified by SYPRO Orange (Sigma #S5692) staining of samples run on sodium dodecyl sulphate-polyacrylamide gel electrophoresis prior to addition of BSA for storage. All quantitations were done using FIJI software analysis of digital photographs (Schindelin et al., Nat Methods 9, 676 (2012)).

Nucleic Acid Preparation

Oligos containing 28S R2 target DNA, non-target (non-specific) DNA, and R2 sequences were ordered from Sigma-Aldrich. The upstream (28Su) and downstream (28Sd) target DNA designations are relative to the R2 insertion dyad within the 28S rRNA gene. The oligo sequences are reported in Table 1.

All the linear DNAs were 50 bp in length. Each arm of most of the three-way and four-way junctions were 25 bp in length except for junctions tested for cDNA synthesis, for which the 28S DNA arm lengths were strategically varied to observe second-strand syntheses products. Diagrams of the constructs are provided in the main figures. Oligos with 28Sd sequence contained either 25 bp or 47 bp of post R2 insertion site 28S rDNA. Seven base pairs of upstream sequence were also included in these “downstream” oligos to span the insertion site. Oligos with 28Su sequence contained 72 bp prior to the insertion site as well as 5 bp of post R2 insertion site 28S rDNA. The largest oligo contained 72 bp of upstream and 47 bp of downstream 28S rDNA. Several oligos incorporated 25 bp of sequence complementary to either the 3′ or the 5′ RNA. Shorter oligos (25 bp) of sequence corresponding to the first and last 25 bp of R2Bm were also used in many of the constructs. The sequence for the x, h, b, and r strands of the nonspecific 4-way junction were obtained from Middleton et al (Middleton and Bond, Nucleic Acids Res 32, 5442 (2004)). The constructs were formed by annealing the component oligos procedure: 20 pmole of the labeled oligo was mixed with 66 pmol of each cold oligo. The primers were annealed in SSC buffer (15 mM sodium citrate and 0.15 M sodium chloride) for 2 minutes at 95° C., followed by 10 minutes at 65° C., 10 minutes at 37° C. and finally 10 minutes at room temperature. One of component oligos had been 5′ 32P end labeled, prior to annealing the other component oligos. The annealed junctions were purified by polyacrylamide gel electrophoresis, eluted in gel elution buffer (0.3 M Sodium acetate, 0.05% SDS and 0.5 mM EDTA pH 8.0), chloroform extracted, ethanol precipitated, and resuspended in Tris-EDTA. Junctions that shared a common labeled oligo were equalized by counts DNA, otherwise equal volumes of purified constructs were generally used in R2 reactions. R2 3′ PBM RNA (249 nt), 5′ PBM RNA (320 nt), and a non-specific RNA (180 nt) were generated by in vitro transcription as previously published (Gasior, et al., J Mol Biol 357, 1383 (2006)).

R2Bm Reactions and Analysis

R2 protein and target DNA binding and cleavage reactions were performed largely as previously reported (Govindaraju, et al., Nucleic Acids Res 44, 3276 (2016)). Briefly, each DNA construct was tested for its ability to bind to R2 protein and to undergo DNA cleavage in the presence and absence of 5′ PBM RNA, 3′ PBM RNA, and non-specific RNA. All the reactions contained excess cold competitor DNA, dIdC. The reactions were loaded onto electrophoretic mobility shifting assays (EMSA) gels and companion denaturing gels for analysis. The ability to bind to branched and linear DNA was obtained from the EMSA gels and the ability to cleave DNA, as well as cleavage position, were obtained from the denaturing urea gels. A+G ladders were run alongside the reactions in the denaturing gels to aid in mapping cleavages. Second-strand synthesis assay was performed by the addition of dNTPs to the DNA cleavage reactions in the absence of RNA. All gels were dried, exposed to a phosphorimager screen, and scanned using a phosphorimager (Molecular dynamics STORM 840). The resulting 16-bit TIFF images were linearly adjusted so that the most intense bands were dark gray. Adjusted TIFF files were quantified using FIJI (Schindelin et al., Nat Methods 9, 676 (2012)).

TABLE 1
Table presenting the DNA and RNA oligonucleotides used to build
the linear and junction DNAs. ‘Comp’ stands for complementary strand.

| Oligo Name | Sequence |
|---|---|
| b-strand | CCTCGAGGGATCCGTCCTAGCAAGCCGCTGCTACCGGAAGCTTCTGGACC |
| | (SEQ ID NO: 1) |
| h-strand | GGTCCAGAAGCTTCCGGTAGCAGCGAGAGCGGTGGTTGAATTCCTCGACG |
| | (SEQ ID NO: 2) |
| r-b strand | CGTCGAGGAATTCAACCACCGCTCTCGCTGCTACCGGAAGCTTCTGGACC |
| | (SEQ ID NO: 3) |
| Pre-cleaved r-b | 1) CGCTGCTACCGGAAGCTTCTGGACC (SEQ ID NO: 4) |
| | 2) CGTCGAGGAATTCAACCACCGCTCT (SEQ ID NO: 5) |
| r-strand | CGTCGAGGAATTCAACCACCGCTCTTCTCAACTGCAGTCTAGACTCGAGC |
| | (SEQ ID NO: 6) |
| x-strand | GCTCGAGTCTAGACTGCAGTTGAGAGCTTGCTAGGACGGATCCCTCGAGG |
| | (SEQ ID NO: 7) |
| h-x strand | GGTCCAGAAGCTTCCGGTAGCAGCGGCTTGCTAGGACGGATCCCTCGAGG |
| | (SEQ ID NO: 8) |
| bₘ-strand | CCTGCAGTGATCCGTCCTAGCAAGCCGCTGCTACCGGAAGCTTCTGGACC |
| | (SEQ ID NO: 9) |
| rₘ-strand | CGTCGAGGAATTCAACCACCGCTCTTCTCACCGATAAGTACGACTCGAGC |
| | (SEQ ID NO: 10) |
| xₘ-strand | GCTCGAGTCGTACTTATCGGTGAGAGCTTGCTAGGACGGATCACTGCAGG |
| | (SEQ ID NO: 11) |
| Ns/28Sd 25 bp | TCCAGAAGCTTCCGGTAGCTTAAGGTAGCCAAATGCCTCGTCATCTAATT |
| | (SEQ ID NO: 12) |
| Comp ns/28Sd 25 bp | AATTAGATGACGAGGCATTTGGCTACCTTAAGCTACCGGAAGCTTCTGGA |
| | (SEQ ID NO: 13) |
| Pre-cleaved comp ns/28Sd | 1) AATTAGATGACGAGGCATTTGGCTA (SEQ ID NO: 14) |
| 25 bp | 2) CCTTAAGCTACCGGAAGCTTCTGGA (SEQ ID NO: 15) |
| xₘ-b strand | GCTCGAGTCGTACTTATCGGTGAGACGCTGCTACCGGAAGCTTCTGGACC |
| | (SEQ ID NO: 16) |
| R2 3′ DNA/ns | TGGCATGATGATCCGGCGATGAAAACCTTAAGCTACCGGAAGCTTCTGGA |
| | (SEQ ID NO: 17) |
| Comp 28Sd 25 bp / Comp | AATTAGATGACGAGGCATTTGGCTATCTCACCGATAAGTACGACTCGAGC |
| R2 3′ DNA | (SEQ ID NO: 18) |
| R2 3′ DNA 25 | TGGCATGATGATCCGGCGATGAAAA (SEQ ID NO: 19) |
| R2 3′ RNA 25 | UGGCAUGAUGAUCCGGCGAUGAAAA (SEQ ID NO: 20) |
| Comp R2 5′ DNA/ comp | AAATTAAAATTATGCGTATCGCCCCCCTTAAGCTACCGGAAGCTTCTGGA |
| 28Sd 25 bp | (SEQ ID NO: 21) |
| R2 5′RNA 25 bp | GGGGCGAUACGCAUAAUUUUAAUUU (SEQ ID NO: 22) |
| R2 3′-5′ DNA | TGGCATGATGATCCGGCGATGAAAAGGGGCGATACGCATAATTTTAATTT |
| | (SEQ ID NO: 23) |
| R2 5′DNA 25 bp | GGGGCGATACGCATAATTTTAATTT (SEQ ID NO: 24) |
| Ns/28Sd 47 bp | TCCAGAAGCTTCCGGTAGCTTAAGGTAGCCAAATGCCTCGTCATCTAATTAGT |
| | GACGCGCATGAATGGATTA (SEQ ID NO: 25) |
| Comp 28Sd 47 bp/comp | TAATCCATTCATGCGCGTCACTAATTAGATGACGAGGCATTTGGCTATTTTCA |
| R2 3′ RNA | TCGCCGGATCATCATGCCA (SEQ ID NO: 26) |
| 28Su 73 bp/ns | GCTCTGAATGTCAACGTGAAGAAATTCAAGCAAGCGCGGGTAAACGGCGGG |
| | AGTAACTATGACTCTCTTAAGGTAGGGTCCAGAAGCTTCCGGTAGCAGCGAG |
| | AGCGG (SEQ ID NO: 27) |
| Comp ns/ comp R2 3′ | CCGCTCTCGCTGCTACCGGAAGCTTCTGGACCCTATTTTCATCGCCGGATCAT |
| RNA | CATGCCA (SEQ ID NO: 28) |
| Comp R2 5′ RNA/ Comp | AAATTAAAATTATGCGTATCGCCCCCCTTAAGAGAGTCATAGTTACTCCCGCC |
| 28Su 73 bp | GTTTACCCGCGCTTGCTTGAATTTCTTCACGTTGACATTCAGAGC (SEQ ID |
| | NO: 29) |
| 28Su 73 bp/28Sd 47 bp | GCTCTGAATGTCAACGTGAAGAAATTCAAGCAAGCGCGGGTAAACGGCGGG |
| | AGTAACTATGACTCTCTTAAGGTAGCCAAATGCCTCGTCATCTAATTAGTGAC |
| | GCGCATGAATGGATTA (SEQ ID NO: 30) |

### Results

Holliday junction resolvases bind to and symmetrically cleave 4-way DNA junctions (Holliday junctions), resolving the junctions into linear DNAs. Holliday junction resolvases recognize DNA structure rather than DNA sequence. The R2 RLE, which shares structural and amino acid sequence homology to Archael Holliday junction resolvases, may exhibit similar DNA binding and cleavage activities.

The potentiality of R2 protein to recognize and bind to a 4-way DNA branched structure was tested by comparing the relative ability of R2 protein to bind to nonspecific linear and nonspecific 4-way junction DNA—individually and in competition (FIG. 2A-2B). The linear and junction DNAs were formed by annealing complementary oligos. The linear and the junction DNA shared a common DNA oligo that had been radioactively labeled prior to annealing. Sharing a common labeled DNA strand allowed radioactive decay counts to be a proxy for equalizing the DNA concentrations between the linear and junction DNAs and for similar DNA sequences to be probed. DNA binding was analyzed by electrophoretic mobility shift assay (EMSA). In the absence of RNA (FIG. 2A-2B), the R2 protein bound to both nonspecific linear and nonspecific 4-way junction DNAs with roughly equal efficiency when individually examined across a protein concentration series. In competitive binding reactions, however, R2 protein had a clear preference for binding to the 4-way junction over the linear DNA. It should be noted that the junction DNA contained a greater number of total base pairs (100 bp; each arm being 25 bp) while the linear DNA was less (50 bp). It is unlikely, however, that the difference in DNA “length” had a significant effect on the observed binding affinity in the competition reaction as the R2 protein did not bind to the linear DNA until most of the junction DNA had been bound: A difference greater than two-fold.

The migration patterns for both linear and junction DNA were quite similar. A portion of the signal was stuck in the well with a smear that ran down from the well to faint protein-DNA complexes in the gel. The gel running protein-DNA complexes for the linear and junction DNAs migrated to roughly the same position within the gel. In the case of the linear DNA the smear continued from well all the way to the free DNA. The migration pattern, particularly that of R2 protein bound to junction DNA, was similar to that of R2 protein bound to its own target DNA in the absence of RNA prior to DNA cleavage (Christensen and Eickbush, Mol Cell Biol 25, 6617 (2005), Christensen and Eickbush, J Mol Biol 336, 1035 (2004)).

In the presence of nonspecific RNA (abbreviated as nsRNA), R2 protein still bound preferentially to junction DNA as it had in the absence of RNA. Again, there was a smear running from the well to the major complex(es) in the gel. The junction and linear protein-RNA-DNA complexes migrated to similar but distinct positions within the gel. In the presence of R2 3′ PBM RNA, R2 protein bound to junction DNA mostly as it did with nonspecific RNA and again 4-way junction DNA was preferred over non-specific linear DNA. Interestingly, in the presence of 5′ PBM RNA the behavior was different (see next section).

### Example 2: 5′ PBM RNA, but not 3′ PBM RNA, is Inhibitory to Binding a Nonspecific 4-Way DNA Junction

An assay was designed to directly compare R2 protein bound to 4-way junction DNA across a range of RNA concentrations for nonspecific RNA, 3′ PBM RNA, and 5′ PBM RNA. For each RNA titration set, the amount of protein used was sufficient to bind most of the junction DNA in the reaction that lacked RNA. In general, the addition of any of the three RNAs pulled material out of the well and into the gel. The R2 RNAs were more efficient at pulling material out of the well and into the gel. A similar phenomenon is observed when R2 protein is bound to its normal (linear) 28S target DNA in the presence of R2 RNA (Christensen and Eickbush, Mol Cell Biol 25, 6617 (2005), Christensen and Eickbush, Proc Natl Acad Sci USA 103, 17602 (2006), Christensen and Eickbush, J Mol Biol 336, 1035 (2004)). Unlike binding to linear 28S target DNA, the presence of 5′ PBM RNA greatly inhibited the binding of R2 protein to the 4-way junction DNA. Only the presence of 5′ PBM RNA greatly affected the binding of R2 protein to junction DNA and inhibition scaled with 5′ PBM RNA concentration. Binding to nonspecific linear DNA and 3-way junction was less affected by the presence of 5′ RNA, but still reduced in its presence. This inhibition is not observed if downstream 28S rDNA sequences are present in any of the DNA constructs (Christensen, et al., Nucleic Acids Res 33, 6461 (2005), Zingler, et al., Cytogenet Genome Res 110, 250 (2005)).

### Example 3: The R2 Protein does not Resolve Nonspecific 4-Way Junction DNA

DNA from reactions of R2 protein bound to nonspecific linear and non-specific 4-way junctions across a range of protein concentrations in the absence of RNA, were analyzed for DNA cleavage events by denaturing polyacrylamide gel electrophoresis. Each strand of the junction and linear DNAs was tracked independently for DNA cleavage events by sequentially radiolabeling the 5′ ends of the different DNA strands. A complicated pattern of random low intensity background cleavages occurred particularly in protein excess. A similar phenomenon of background cleavages occurs for R2 protein bound to its normal 28S target DNA in the absence of RNA when R2 protein is in excess. The background cleavages on the non-specific junction were not structure driven as the cleavages occurred in identical positions in the linear DNA of the same sequence. The presence of any of the three RNAs (5′ PBM RNA>3′ PBM RNA>nonspecific RNA) abolished the random background DNA cleavage.

### Example 4: Linear Target DNA and TPRT Product are Poor Substrates for Second-Strand Cleavage

R2Bm inserts into a specific site in the 28S rDNA. It was determined that the protein subunit bound to target sequences downstream of the insertion site provides the endonuclease involved in second-strand (i.e., top-strand) DNA cleavage. Second-strand cleavage, however, has always been tricky to achieve and study. Previously, second-strand cleavage neeeded a narrow range of 5′ PBM RNA, R2 protein, and DNA ratios. The prior data indicated that first-strand DNA cleavage is probably needed before the second-strand can be cleaved and that the downstream subunit must be bound to the DNA (which needed 5′ PBM RNA), and that the 5′ PBM RNA must then dissociate from the downstream subunit for second-strand cleavage to occur. In vivo, with a full length R2 RNA, the process of TPRT would be believed to pull the 5′ PBM RNA from the downstream subunit putting the downstream subunit into the “no RNA bound” state and thus initiating second-strand DNA cleavage.

Given the R2 protein is able to bind branched DNAs in the absence of RNA, the role of DNA structure on the downstream subunit's ability to cleave DNA in the absence of RNA was investigated. The DNA constructs contained the binding site for the downstream R2 protein subunit but not binding site for the upstream-binding R2 protein subunit in order to isolate activities associated with the downstream subunit. The upstream DNA sequence was replaced by non-specific DNA derived from the 4-way junction used in the previous figures. Linear DNAs containing downstream 28S DNA were not substrates for second strand cleavage regardless of the presence or absence of a first strand DNA cleavage event (FIG. 2, constructs iii, and iv). Neither was a post-TPRT analog (construct v) able to be cleaved by the R2 protein. The TPRT analog was a 3-way junction containing downstream 28S DNA that was precleaved at the first (bottom) strand cleavage site and covalently linked to cDNA sequences corresponding to the 3′ end of the R2 element, as would be thought from a TPRT reaction. Annealed to the cDNA portion of the construct was either 25 bp of R2 RNA or a DNA version of the same 25 bp. The R2Bm protein was unable to cleave the top-strand of these 3-way junctions. It did not matter if the R2 3′ sequence containing arm was in the form of an RNA-DNA duplex or a DNA duplex.

### Example 5: Specific 4-Way Junction(s) are Cleaved by R2 Protein

Unlike the linear and TPRT-junction (FIG. 3, constructs iii-v) DNAs, a 4-way junction that included target sequence and R2 sequences was found to be cleavable by R2 protein (FIG. 3, construct viii). Construct viii was similar to the TPRT-j unction (construct v) but with an additional arm: the 5′ R2 arm. Both the R2 5′ arm and the R2 3′ arm were 25 bp in length and consisted of a RNA-DNA duplex. Construct viii mimics a proposed association between the cDNA and the target DNA. The 5′ end of the R2Bm mRNA is believed to contain rRNA sequence corresponding to the upstream target DNA (Eickbush, et al., PLoS One 8, e66441 (2013), Stage and Eickbush, Genome Biol 10, R49 (2009), Fujimoto et al., Nucleic Acids Res 32, 1555 (2004), Eickbush, et al., Mol Cell Biol 20, 213 (2000)). The reverse transcribed cDNA could then hybridize to the top strand of the target to form the 4-way junction. A completely covalently closed all DNA version of the same junction was also able to be cleaved, albeit to a lesser degree (see construct vi, FIG. 3) as was a construct lacking the R2 3′ arm (construct vii).

### Example 6: Further Exploration of Second-Strand DNA Cleavage

To further explore the structure requirements for second-strand cleavage, a number of structural-variants (i.e., partial-junctions) of FIG. 3 construct viii were tested for cleavability (FIGS. 4A-4B, constructs i-viii). FIG. 3 construct viii is identical to FIG. 4A construct i except that the 28S downstream arm was increased to 47 bp in length instead of the original 25 bp used in FIG. 3 construct viii. This adjustment was to set the downstream DNA in the FIG. 4A-4B constructs equal to the amount of downstream DNA included in historical linear DNA constructs used in previous publications (Govindaraju, et al., Nucleic Acids Res 44, 3276 (2016)). The reason for testing the cleavability of partial junctions (FIG. 4A-4B, junctions ii-viii) was to determine to what extent, if any, the DNA cleavage signal observed in FIG. 3 may have been coming from the minor, but present, contaminating partial junctions in the binding and cleavage reactions. It was also to determine if constructs mimicking cellular removal of the RNA component (e.g., by cellular RNases; construct vi-viii) faired better or worse at being cleaved by the R2 protein than constructs with intact RNA-DNA duplexes. It appears that several of the partial junctions (complexes ii and iii) can be cleaved and thus likely partially contribute the overall cleavage in reactions containing the full junction (complex i). The 4-way junction that lacked both RNA components (complex vi) was nearly uncleavable indicating the need for double stranded R2 arms. The 4-way junction that lacked the 5′ end RNA but contained the 3′ end RNA; construct vii) also failed to appreciably cleave indicating the importance of the presence a RNA-DNA duplex in R2 5′ arm. The 4-way junction that lacks the 3′ end RNA but contained the 5′ end RNA (construct viii) cleaved well. Indeed, it was more efficiently cleaved than construct i indicating that the presence of duplex in the R2 3′ arm is partially inhibitory but that the presence of duplex in the 5′ arm is stimulatory.

In order to investigate the relative importance of upstream target sequences on second-strand DNA cleavage, 73 bp of upstream 28S DNA was incorporated into the 4-way junction FIG. 4C-4D; constructs ii-iv). In construct ii the 47 bp of downstream 28S DNA was replaced with nonspecific DNA and construct iii contained the full target DNA sequence (73 bp of upstream 28S DNA and 47 bp of downstream 28S DNA). Construct ii was able to be cleaved, albeit much less efficiently that construct i which contained the downstream target DNA but not upstream as in previous figures. The fact that construct ii is able to be cleaved indicates that perhaps the 12 bp (7 bp of upstream and 5 bp of downstream DNA) common to both constructs i and ii might be involved in helping to direct DNA cleavage. Paradoxically, construct iii, which contains the full target sequence, was less efficient at being cleaved than even construct ii. Adding the flap, or displaced strand (construct iv), thought to occur during template jumping noticeably increased cleavability of the junction.

### Example 7: Second-Strand Cleavage Leads to Second-Strand Synthesis in the Presence of dNTPs

To test if second-strand cleavage could progress to second-strand synthesis dNTPs were added to the DNA cleavage reaction. The construct used to test for second-strand synthesis was construct i of FIG. 4A-4B. It cleaved relatively well. A range of R2 protein concentrations was used and the reactions were analyzed by denaturing (FIG. 5) and native polyacrylamide gel electrophoresis. The labeled strand of the 4-way junction was 72 nt uncleaved and 24 nt in length upon second-strand DNA cleavage (marked as SSC on the denaturing gel). Second-strand synthesis (SSS), i.e., extension of the labeled strand post DNA cleavage, would generate a 50 nt product when analyzed on a denaturing gel. Second-strand DNA synthesis was observed only at the higher end of the protein titration series in the denaturing gels. The reason for this becomes clear in the native (EMSA) gels. Upon cleavage, the 4-way junction is resolved into two linear DNAs: one DNA containing the downstream and R2 3′ arms and one DNA containing the “upstream” and R2 5′ arms. The R2 protein appeared to remain bound to the DNA that contained the downstream 28S DNA after DNA cleavage while DNA with the DNA containing the non-specific “upstream” DNA was released. The release DNA primer-template is extended by the R2 RT only when protein is in excess. The migration positions of product of second-strand cleavage and second-strand synthesis is indicated next to the EMSA gels.

The signal above full length oligo on the denaturing gels in the presence of dNTPs results from the original full-length oligo being extended by R2. R2 can take almost any 3′ end and extend it given a template in cis or in trans (Bibillo, et al., J Biol Chem 279, 14945 (2004), Bibillo and Eickbush, J Mol Biol 316, 459 (2002)).

### Example 8: Second-Strand Synthesis on Precleaved DNA Constructs

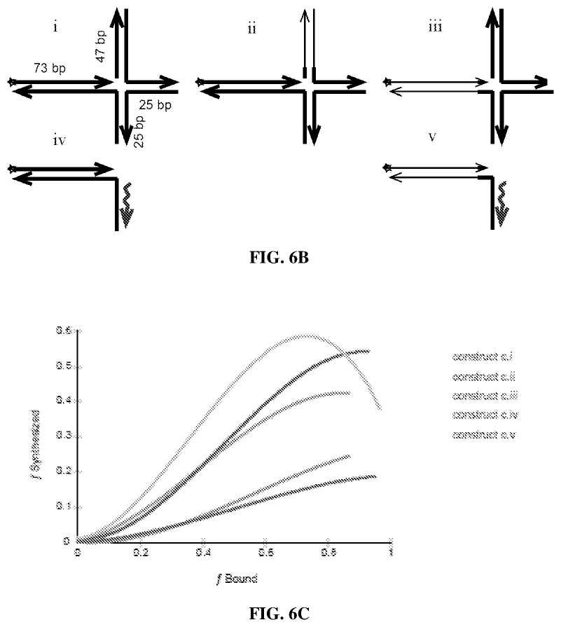

Although the primer-template is released from the protein-DNA complex when the upstream DNA is not present in the 4-way junction, one might think that this would not occur in vivo with in a junction that contained the full target sequence. In part, this belief is because it is believed that the downstream subunit performs second-strand synthesis (Christensen and Eickbush, Mol Cell Biol 25, 6617 (2005)). Unfortunately, junctions with full target sequence do not cleave well (FIGS. 4C and 4D) and second-strand synthesis is below the detection level when tested in vitro. For this reason, a post-second strand cleavage analog was generated. In order to keep the second-strand cleavage products tethered together, the R2 3′ and 5′ end “RNAs” were covalently linked, although instead of RNA DNA was used for convenience. The upstream 28S DNA containing second-strand cleavage product was able to undergo primer extension (i.e., second-strand synthesis) in the tethered configuration. The 5′ end cDNA strand was used as the template (FIG. 6A).

In order to determine which R2 protein subunit is used for second-strand synthesis, linear (FIG. 6B, complexes iv and v) and tethered (FIG. 6B, complexes i and iii) post-second strand cleavage products were tested for their relative ability to undergo second-strand synthesis (FIG. 6C). The results are consistent with the subunit bound to the 4-way junction being responsible for second-strand cleavage. Complex iii was the most efficient substrate for second-strand synthesis and complex was the least efficient substrate.

### Example 9: Mutations in the Core Residues of the HINALP and CCHC Motifs Affect Target DNA Binding and Leads to Loss of DNA Cleavage Specificity

### Materials and Methods

Mutations

To investigate the role of the linker region's presumptive α-finger (HINALP motif region), and zinc knuckle (CCHC motif region), a number of double point mutants were generated (FIG. 8B). The mutations in the presumptive α-finger region included GR/AD/A, VH/ATH/A, H/AIN/ALP, SR/AIR/A and SR/AGR/A. The H/AIN/AALP and SR/AIR/A mutations resulted in a reduction of soluble protein being recovered compared to wild type (WT) protein. The VH/ATH/A mutation did not produce soluble protein and was dropped from the study. The mutations in the zinc knuckle region were C/SC/SHC, CR/AAGCK/A, E/AT/AT, HILQ/AQ/A and RT/AH/A (FIG. 8B). The C/SC/SHC mutation resulted in greatly reduced soluble protein being recovered compared to wild type (WT) protein. The E/AT/AT mutation did not yield usable quantities of protein and was dropped from the study.

Protein and Nucleic Acid Preparations

Protein was expressed and purified as previously published (Govindaraju, et al., Nucleic Acids Res. 44, 3276-3287 (2016)). A QuikChange site-directed mutagenesis kit (Stratagene #200523-5) was used to generate the GR/AD/A, SR/AIR/A, SR/AGR/A, H/AIN/ALP, C/SC/SHC, CR/AAGCK/A, HILQ/AQ/A and RT/AH/A mutants. 5′ PBM (320 nt), 3′ PBM (249 nt), linear target DNA, and 4-way junction were prepared as previously published (Govindaraju, et al., Nucleic Acids Res. 44, 3276-3287 (2016)).

R2Bm Reactions and Analysis

DNA binding, first and second strand cleavage, and first and second strand synthesis reactions were performed as previously reported (Govindaraju, et al., Nucleic Acids Res. 44, 3276-3287 (2016)).

For DNA binding assays, a mastermix containing all the components except for the protein was made and aliquoted. The binding reaction was initiated by adding 3u1 of protein at the known and equalized concentrations across all proteins being tested in a data set. Duplicate reactions were prepared for each data set and two different data sets were generated, each at a different protein concentrations. WT and WT KPD/A proteins acted as binding activity references and positive controls for endonuclease active and endonuclease deficient mutations, respectively.

For DNA cleavage assays, a master mix containing all the components except protein and DNA was made and aliquoted. Protein from protein dilution series was allowed to bind to RNA for 5 minutes at 37° C. prior to adding the target DNA to start the cleavage reaction. The reaction was incubated for 30 minutes at 37° C. The reactions were kept on ice before running on 5% native (1× Tris-borate-EDTA) polyacrylamide gels and on denaturing (8M urea) 7% polyacrylamide gels.

First and second strand synthesis reactions contained labelled target DNA in the master mix along with all other components except for protein. Pre-cleaved linear DNA was used so that mutants deficient in DNA cleavage could be tested along with mutants with normal cleavage ability. Target DNA substrate for second strand synthesis assay was a four-way junction DNA pre-cleaved at the second strand and is described in Chapter 2. Similar to the cleavage assay the reactions were analyzed by both native and denaturing polyacrylamide gels.

All gels were dried and quantitated using a phosphorimager (Molecular dynamics STORM 840) and FIJI (Schindelin, et al., Nat. Methods (2012). doi:10.1038/nmeth.2019.Fiji).

### Results

There were four double point mutants created in the HINALP region and four in the zinc knuckle region. The H/AIN/AALP and the C/SC/SHC mutants appear to have nearly identical phenotypes. Both sets of mutations severely impair DNA binding to the linear DNA as well as the ability to form the correct DNA-RNA-Protein complexes in EMSA gels on linear DNA

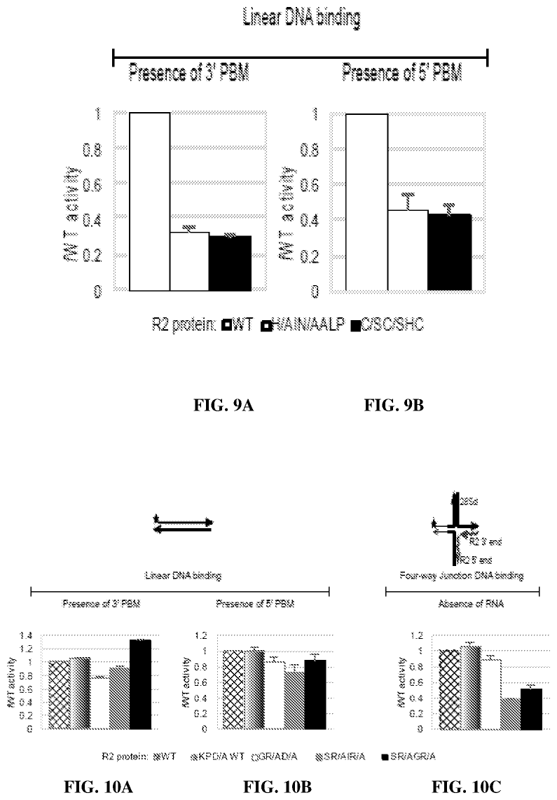

(FIG. 9A-9B). Only the well complex and a diffuse smear leading down from the well to the free DNA are observed (FIG. 9A-9B). This observation is true for both upstream binding conditions (i.e., presence of 3′ PBM RNA) and downstream binding conditions (i.e., presence of 5′ PBM RNA). The Cysteine and Histidine residues of the zinc knuckle motif are the presumptive zinc coordinating residues. The C/SC/SHC mutation may promote local misfolding of the linker. The H/AIN/AALP mutation may have also affected the folding of the linker.

In the presence of 3′ PBM RNA, the H/AIN/AALP and C/SC/SHC mutants showed little to no first strand cleavage at the insertion site. Second-strand DNA cleavage was similarly abolished in the presence of 5′ PBM RNA. Instead of site specific DNA cleavage abundant promiscuous cleavages were observed at aberrant sites on both strands of the target DNA.

### Example 10: Mutations in the Presumptive α-Finger Affect DNA Binding, Especially to a Specific Branched Integration-Intermediate Analog

To better determine if the presumptive α-finger is involved in securing protein to upstream and/or downstream target DNA sequences, mutations surrounding the core HINALP motif were tested. The GR/AD/A, SR/AIR/A and SR/AGR/A mutants were tested for their ability to bind linear target in the presence of 3′ PBM RNA and in the presence of 5′ PBM RNA. Two positive controls were used, WT R2 protein and R2 protein with a catalytic residue of the RLE mutated to alanine (KPD/A) so as to knockout DNA cleavage but not DNA binding so that the α-finger mutations that either do or do not affect DNA cleavage (see the next section) are appropriately controlled for. The DNA binding ability of the mutant relative to the control R2 proteins were assayed using Electrophoretic Mobility Shift Assays (EMSAs) (FIG. 10A-10B). Duplicate lanes were loaded and duplicate binding reactions were run. Vector control extract and no protein lanes served as negative control lanes.

Upstream target DNA binding was moderately reduced (24%) by the GR/AD/A mutation and very mildly reduced (13%) by the SR/AIR/A mutation. But upstream target DNA binding activity was significantly increased up to 32% by SR/AGR/A mutant (FIG. 10A-10B). Downstream target DNA binding activity for GR/AD/A and SR/AGR/A mutants was similar to WT activity, with only a mild decrease of −13%. The SR/AIR/A mutation decreased binding in the range of 19-28%. All the three mutants did not seem to affect the migration pattern of protein-RNA-DNA complexes much if at all, although, more of the well complex formation was observed for SR/AIR/A mutant (FIG. 10A-10B). The ability of the mutants to bind to linear target DNA in the absence of RNA is presented in FIG. 10D. The ability of the mutants to bind a four-way junction integration intermediate was also tested. The four-way junction mimics the branched structure adopted by 28S rDNA after the template jump step, and contains 28Sd rDNA sequence (north arm), a non-specific sequence (west arm), a R2 5′-end RNA-DNA duplex (south arm), and a R2 3′-end RNA-DNA duplex (east arm) (FIG. 10C) (see also, Example 1-8). The four-way junction DNA was radiolabeled at the top strand of the 5′ end of the west arm. The junction DNA was incubated with R2 protein in the absence of RNA and aliquots were run in EMSA gel (FIG. 10C). After quantitation as described above, the two mutants were shown to have significantly reduced the ability of R2 protein to bind to the four-way junction, SR/AIR/A by 63% and SR/AGR/A by 48% while GR/AD/A mutant's binding activity was comparable to that of WT activity showing only a mild reduction of 12%.

### Example 11: Mutations in the Presumptive α-Finger Reduce First-Strand DNA Cleavage

The ability of the GR/AD/A, SR/AIR/A and SR/AGR/A mutants to perform first-strand DNA cleavage was assayed. The R2 proteins were prebound to 3′ PBM followed by incubation with target DNA. A protein titration series was used (seven 1:3 protein dilutions). An aliquot of each reaction was run on a EMSA gel and on a denaturing (8M urea) polyacrylamide gel. The target DNA was ³²P labeled at the 5′ end of the bottom strand (i.e., 28S antisense strand) so that the cleavage of this strand could be tracked in the denaturing gel.

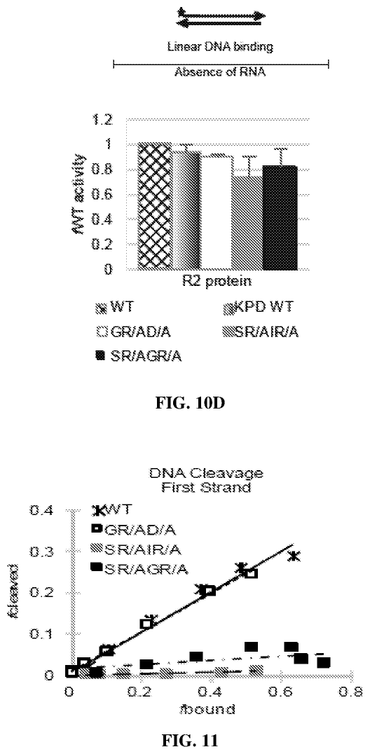

At higher protein concentration lanes (first two) in EMSA gel, Protein-DNA complexes corresponding to the one seen in the absence of RNA were observed for WT, GR/AD/A and SR/AGR/A mutants as the RNA concentration had been held constant and as protein neared parity with the RNA concentration, DNA-complexes appeared along with protein-RNA-DNA complexes before everything becomes stuck in the wells. The mutations did not appear to greatly affect the migration pattern of protein-RNA-DNA complexes as compared to WT. The cleavage activity of each of the mutant is reported as a scatter plot of the fraction of cleaved DNA (fcleaved), calculated from the urea denaturing gels, as a function of the fraction of bound (fbound) DNA, calculated from the EMSA gels. GR/AD/A mutant did not affect the first strand cleavage activity of R2 protein, however, the SR/AIR/A and SR/AGR/A mutants significantly reduced the ability of the bound protein to undergo first strand DNA cleavage (FIG. 11). No cleavages beyond the R2 cleavage site were observed for either WT or mutants.

### Example 12: Mutations in the Presumptive α-Finger Reduce First Strand cDNA Synthesis

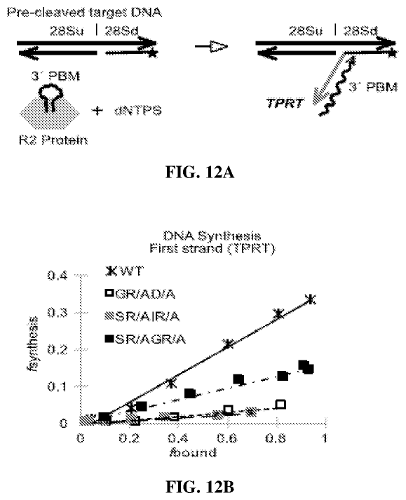

To investigate if HINALP region affects TPRT (first-strand DNA synthesis), pre-cleaved target DNA with nick at the insertion site on first/bottom strand was incubated with R2 protein in the presence of 3′ PBM RNA and dNTPs (FIG. 12A). The target DNA was radiolabeled at the 5′ end of the bottom strand to track the formation of the TPRT product. Aliquots of reactions across a protein titration series were assayed on EMSA and denaturing polyacrylamide gels. A graph of the fraction of target DNA that underwent TPRT (fsynthesis) as a function of fraction of target DNA bound by R2 protein (fbound) is reported in FIG. 12B. GR/AD/A and SR/AIR/A mutants completely abolished the TPRT activity while SR/AGR/A mutant reduced first strand synthesis activity by approximately 50% (FIG. 12B).

### Example 13: Mutations in the Presumptive α-Finger Affect Second-Strand DNA Cleavage

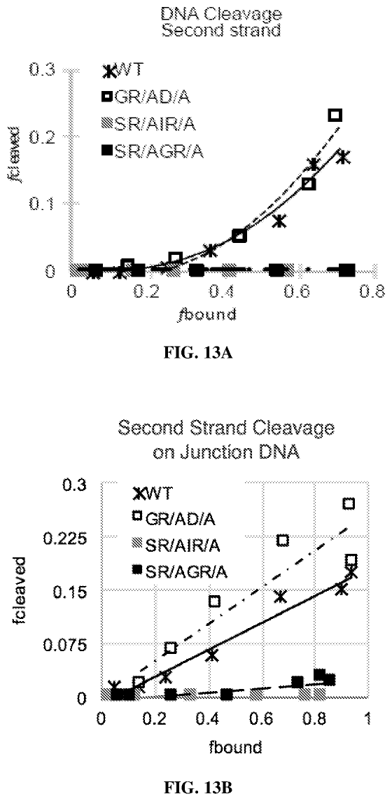

In order to determine the role, if any, the GR/AD/A, SR/AIR/A and SR/AGR/A mutants have on second-strand cleavage, two different cleavage assays were undertaken: (1) on linear target DNA in the presence of 5′ PBM RNA, and (2) cleavage on 4-way junction DNA in the absence of RNA. On linear DNA, R2 protein binds downstream of the insertion site in the presence of 5′ PBM RNA but only cleaves once the RNA dissociates from the complex. The dissociation occurs as the RNA to protein ratio drops across the protein titration series (RNA is held constant) (Christensen, et al., Proc. Natl. Acad. Sci. U.S.A 103, 17602-17607 (2006)). In EMSA gel, the migration pattern of protein-RNA-DNA complexes of mutants were similar to that of WT, however, a band corresponding to a second strand cleaved product located immediately below the major protein-RNA-DNA complex was absent for SR/AIR/A and SR/AGR/A mutants. In denaturing gel, the signal for second strand cleaved product was not visible for SR/AIR/A and SR/AGR/A mutants. Non-specific cleavages were not observed for any of the mutants. While GR/AD/A showed WT activity, SR/AIR/A and SR/AGR/A mutants knocked out the endonuclease activity of R2 protein to make second strand cleavage on linear target DNA (FIG. 13A).

As noted above, second strand cleavage activity was also tested using a 4-way junction integration intermediate (FIG. 13B). Second strand DNA cleavage is believed to occur when the protein is in the “no RNA” bound state¹⁶ and that the proper substrate for DNA cleavage is a 4-way junction intermediate formed by template jump. A diagram of the junction DNA used is shown in FIG. 10C. The junction DNA was radiolabeled at the 5′ end of the west arm to track cleavages on the top strand of the 28S DNA. The cleavage activity for mutants was tested against WT as indicated in the previous target DNA cleavage assays but in the absence of RNA. Endonuclease activity to cleave the second strand on a four-way junction DNA was completely knocked out by SR/AIR/A and SR/AGR/A mutants while GR/AD/A mutant showed WT cleavage activity or better as shown in the scatterplot (FIG. 13B).

### Example 14: Mutations in the Presumptive α-Finger Affect Second Strand Synthesis

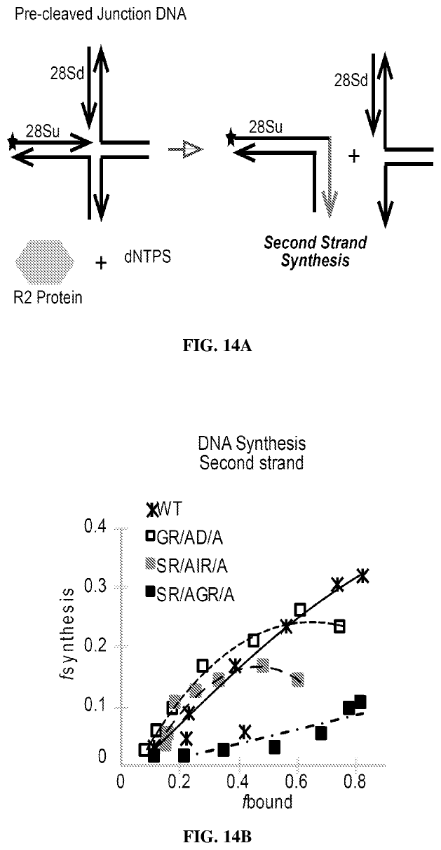

In addition to testing second strand cleavage activity of HINALP mutants, the same mutants were subjected to experiments designed to test second stand DNA synthesis activity. As DNA cleavage is not very efficient, pre-cleaved DNA was used, and as the upstream and the downstream ends separate in vitro post DNA cleavage, the two ends were held together by a covalent linkage between the east and south arms (i.e., between R2 5′ end sequence and R2 3′ end sequence) (see diagram in FIG. 14A-14B) (see also Examples 1-8). This post second-strand cleavage analog was developed and reported in a previous study. The HINALP mutants were tested for second-strand DNA synthesis activity using this construct (FIG. 14C). The 5′ end of the west arm was radiolabeled to visualize the newly synthesized second-strand in denaturing gel (represented by black star in FIG. 14A-14B). The graph shown in FIG. 14C was obtained from EMSA and denaturing gels as described previously for first strand synthesis assay. GR/AD/A mutant seems to act more like WT except that at the highest protein concentration, the amount of second strand synthesis goes down. SR/AIR/A mutant looks more like WT until about 40% of the target DNA is protein-bound but with increasing protein concentrations, the second strand synthesis decreases significantly. SR/AGR/A mutant drastically diminishes the ability of R2 protein to synthesize second strand as shown in the FIG. 14C graph.

### Example 15: Mutating Residues in the Zinc Knuckle Region Affect Target DNA Cleavage and Second Stand Synthesis

While C/SC/SHC mutant showed to affect target DNA binding and cleavage, the role of CCHC region was further investigated with the help of three additional double point mutants in this region: CR/AAGCK/A, HILQ/AQ/A and RT/AH/A (FIG. 8B). The mutants were assayed for DNA cleavage and new strand synthesis activities as described previously.

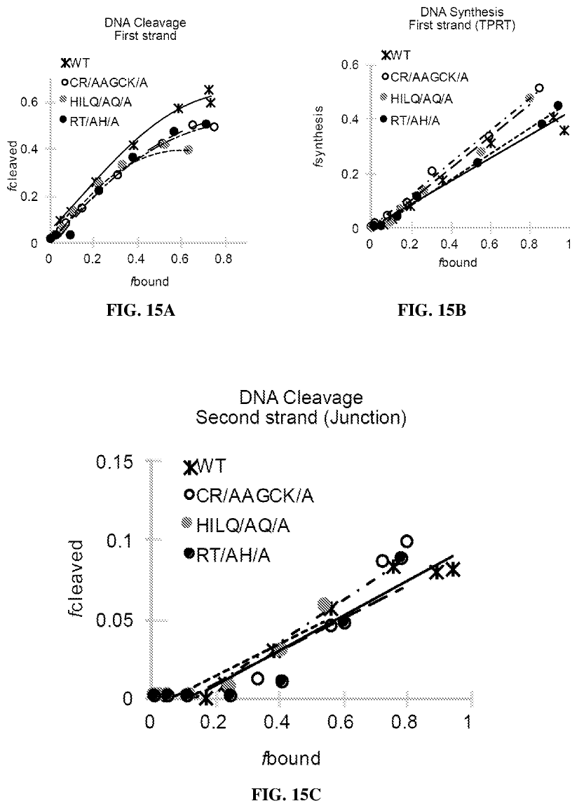

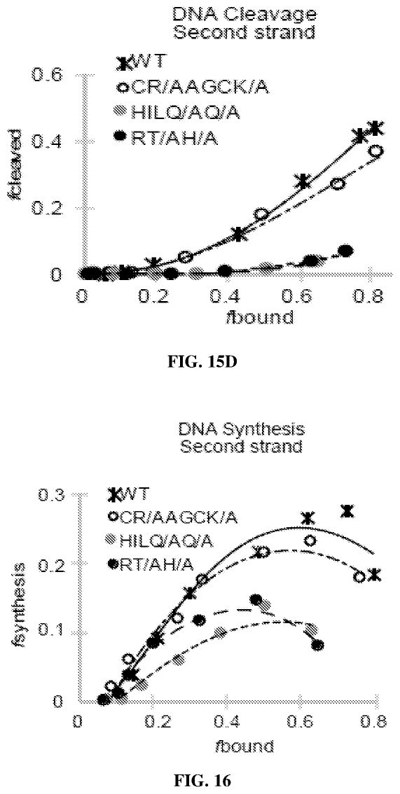

All the three mutants only slightly reduced the ability of the R2 protein to cleave the first strand at the insertion site (FIG. 15A), and they did not seem to have any effect on the first strand synthesis activity by TPRT (FIG. 15B). Although CR/AAGCK/A, HILQ/AQ/A and RT/AH/A mutants were nearly WT for first strand cleavage and synthesis, at least two of the mutants, HILQ/AQ/A and RT/AH/A significantly abolished second strand cleavage activity on a linear DNA (FIG. 15D). In addition to the decrease in second strand cleavage activity at the insertion site, the endonuclease of RT/AH/A mutant was also found to be cleaving at a nearby site on top strand of linear target. The second strand cleavage activity of the mutants were also tested using the four-way junction target DNA, however, all the three mutants showed WT activity (FIG. 15C). Yet again, the endonuclease of RT/AH/A mutant showed an additional cleavage at a non-R2 specific site.

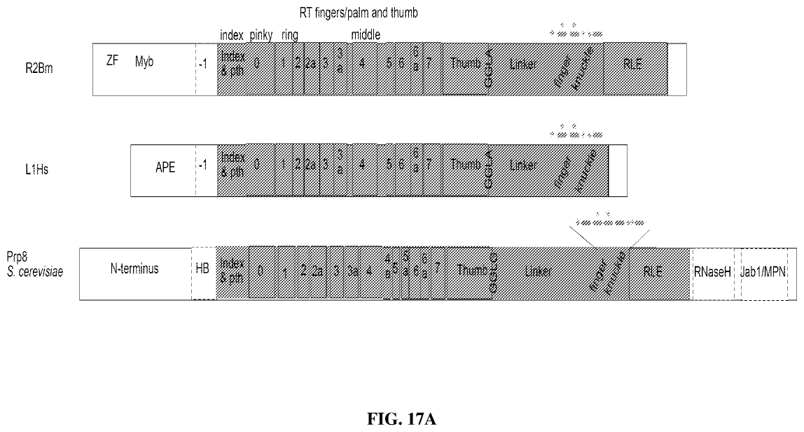

Second strand synthesis assay with a pre-nicked four-way junction DNA, as shown in FIG. 14, was conducted for the three CCHC region mutants as described before for HINALP region mutants. The second strand synthesis product formation per bound unit of target DNA for CR/AAGCK/A looked very similar to that of WT, but for HILQ/AQ/A and RT/AH/A there was huge reduction in second strand synthesized product formation as shown in FIG. 16.

TABLE 2
Summary of DNA binding, cleavage, and synthesis results.

| | Linear | | | | | Junction | | | either |
|---|---|---|---|---|---|---|---|---|---|
| | DNA | First | First | DNA | Second | | Second | Second | Non-R2 |
| | binding | strand | strand | binding | strand | DNA | strand | strand | site |
| | (3' PBM) | cleavage | synthesis | (5' PBM) | cleavage | binding | cleavage | synthesis | cleavage |
| GR/AD/A | − | WT | ∅ | WT | WT | WT | WT | WT | None |
| SR/AIR/A | WT | ∅ | ∅ | − | ∅ | −−− | ∅ | −− | None |
| SR/AGR/A | ++ | ∅ | −− | WT | ∅ | −− | ∅ | ∅ | None |
| H/AIN/AALP | −−− | ∅ | N.A. | −−− | ∅ | N.T. | N.T. | N.A. | Yes |
| C/SC/SHC | −−− | ∅ | N.A. | −−− | ∅ | N.T. | N.T. | N.A. | Yes |
| CR/AAGCK/A | N.T. | − | WT | N.T. | − | N.T. | WT | WT | None |
| HILQ/AQ/A | N.T. | − | WT | N.T. | ∅ | N.T. | WT | −−− | None |
| RT/AH/A | N.T. | − | WT | N.T. | ∅ | N.T. | WT | −−− | Yes |

Not Applicable (N.A.), Not tested (N.T.)
“++”: +30% and above
“+”: +15% to 30%
“WT”: 15% to −15% of WT activity: functionally WT
“−”: −15% to −30%: modest reduction
“−−”: −30% to −50%: major reduction
“−−−” : −50% to 75%: severe reduced
“∅”: 75% and above: functionally dead

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A RNA component comprising a DNA targeting sequence, one or more protein binding motifs (PBM), and a nucleic acid sequence of interest to be integrated into a DNA target site, wherein the DNA targeting sequence, the protein binding motifs, and sequence of interest are operably linked such that they can bind to a protein component derived from a parental Long Interspersed (LINE) element protein and be reverse transcribed into cDNA and the cDNA can be integrated into the DNA at the DNA target site.

2. The RNA component of claim 1, wherein the protein component comprises one or more of an RNA binding domain, a linker domain, a reverse transcriptase, a DNA endonuclease, and wherein the one or more protein binding motifs bind the RNA component to the RNA binding domain, linker domain, reverse transcriptase, DNA endonuclease, or a combination thereof of the protein component.

3. The RNA component of claim 1, wherein the RNA component comprises elements from or derived from a parental LINE or SINE backbone and the nucleic acid sequence of interest of RNA component is heterologous to the LINE or SINE; wherein protein component comprises elements from or derived from a parental LINE; or a combination thereof, and/or (a) the DNA targeting sequence is heterologous to the parental LINE or SINE; and/or (b) the sequence of interest encodes a gene, a fragment of a gene, or a functional nucleic acid.

4. (canceled)

5. (canceled)

6. The RNA component of claim 1, comprising: (a) the 3′ PBM sequence from or derived from a parental LINE or SINE element; and/or (b) a CRISPR/Cas tracer sequence, a CRISPR/Cas guide sequence, or a combination thereof; and/or (c) a 5′ PBM sequence from or derived from the parental LINE or SINE element; and/or (d) a ribozyme.

7. (canceled)

8. (canceled)

9. The RNA component of claim 6, wherein: (a) the 5′ PBM comprises a non-functional IRES sequence; (b) the ribozyme is Hepatitis Delta Virus like ribozyme; and/or (c) the RLE LINE is an R2 LINE.

10. (canceled)

11. (canceled)

12. The RNA component of claim 2, wherein the parental LINE or SINE is a Restriction-like endonuclease (RLE) LINE.

13. (canceled)

14. The RNA component of claim 3, wherein the parental LINE or SINE backbone of the RNA component and the parental LINE backbone of the protein component are the same LINE and/or the SINE is derived from or an ancestor of the LINE.

15. A protein component comprising a DNA binding domain, an RNA binding domain, a reverse transcriptase, a linker domain, and an endonuclease wherein the DNA binding domain, RNA binding domain, reverse transcriptase, linker domain, and endonuclease are operably linked such that they can bind to an RNA component and DNA at a DNA target site, and facilitate reverse transcription of the RNA component into cDNA, and integration of the cDNA into the DNA at the DNA target site.

16. The protein component of claim 15, wherein the RNA component comprises a DNA targeting sequence, one or more protein binding motifs, and a nucleic acid sequence of interest to be integrated into the DNA target site; and/or (b) the RNA component comprises elements from or derived from a parental LINE or SINE backbone and the nucleic acid sequence of interest of RNA component is heterologous to the LINE or SINE; wherein protein component comprises elements from or derived from a parental LINE; or a combination thereof; and/or the DNA binding domain is mutated relative to the parental LINE DNA binding domain; and/or
(c) the DNA binding domain is substituted with an alternative DNA binding domain relative to the parental LINE DNA binding domain; and/or
(d) the DNA binding domain is substituted with an alternative DNA binding domain relative to the parental LINE DNA binding domain.

17. (canceled)

18. (canceled)

19. (canceled)

20. The protein component of claim 16, wherein: (a) the DNA binding domain is a DNA binding domain from another DNA binding protein; (b) the DNA binding domain comprises one or more of a helix-turn-helix, zinc finger, leucine zipper, winged helix, winged helix-turn-helix, helix-loop-helix, HMG-box, Wor3 domain, OB-fold domain, immunoglobulin fold, B3 domain, TAL effector, or RNA-guided domain; (c) the parental LINE or SINE is a Restriction-like endonuclease (RLE) LINE; or (d) the parental LINE or SINE backbone of the RNA component and the parental LINE backbone of the protein component are the same LINE and/or the SINE is derived from or an ancestor of the LINE.

21. (canceled)

22. The protein component of claim 15 wherein the sequences of one or more of the RNA binding domain, reverse transcriptase, linker domain, and endonuclease are the same as those of the parental LINE element protein, or mutated to improve binding or enzymatic activity for the RNA component relative to the parental LINE element protein.

23. (canceled)

24. The protein component of claim 20, wherein the RLE LINE is an R2 LINE.

25. (canceled)

26. A vector encoding the RNA component of claim 1.

27. A vector encoding the protein component of claim 15.

28. An engineered transposon comprising the RNA component of claim 1.

29. The transposon of claim 28, wherein a productive 4-way junction is formed during the integration reaction at the DNA target site.

30. A pharmaceutical composition comprising the RNA component of claim 1.

31. A method of introducing a nucleic acid sequence of interest into the genome of a cell or cells comprising contacting the cell or cells with the RNA component of claim 1.

32. The method of claim 31, wherein the cells are contacted in vitro; wherein the cells are contacted in vivo; or expression of the nucleic acid sequence of interest in the cells improves a one or more symptoms of a disease or disorder, or a molecular pathway underlying a disease or disorder.

33. The method of claim 32, wherein the cells are subsequently introduced into a subject and optionally, wherein an effective number of cells are modified to treat a subject in need thereof.

34. (canceled)

35. (canceled)

36. (canceled)


SEQUENCE LISTING (44 sequences)

SEQ ID NO: 1 - DNA, 50 bp, Artificial Sequence
  synthetic polynucleotide
cctcgagggatccgtcctagcaagccgctgctaccggaagcttctggacc

SEQ ID NO: 2 - DNA, 50 bp, Artificial Sequence
  synthetic polynucleotide
ggtccagaagcttccggtagcagcgagagcggtggttgaattcctcgacg

SEQ ID NO: 3 - DNA, 50 bp, Artificial Sequence
  synthetic polynucleotide
cgtcgaggaattcaaccaccgctctcgctgctaccggaagcttctggacc

SEQ ID NO: 4 - DNA, 25 bp, Artificial Sequence
  synthetic polynucleotide
cgctgctaccggaagcttctggacc

SEQ ID NO: 5 - DNA, 25 bp, Artificial Sequence
  synthetic polynucleotide
cgtcgaggaattcaaccaccgctct

SEQ ID NO: 6 - DNA, 50 bp, Artificial Sequence
  synthetic polynucleotide
cgtcgaggaattcaaccaccgctcttctcaactgcagtctagactcgagc

SEQ ID NO: 7 - DNA, 50 bp, Artificial Sequence
  synthetic polynucleotide
gctcgagtctagactgcagttgagagcttgctaggacggatccctcgagg

SEQ ID NO: 8 - DNA, 50 bp, Artificial Sequence
  synthetic polynucleotide
ggtccagaagcttccggtagcagcggcttgctaggacggatccctcgagg

SEQ ID NO: 9 - DNA, 50 bp, Artificial Sequence
  synthetic polynucleotide
cctgcagtgatccgtcctagcaagccgctgctaccggaagcttctggacc

SEQ ID NO: 10 - DNA, 50 bp, Artificial Sequence
  synthetic polynucleotide
cgtcgaggaattcaaccaccgctcttctcaccgataagtacgactcgagc

SEQ ID NO: 11 - DNA, 50 bp, Artificial Sequence
  synthetic polynucleotide
gctcgagtcgtacttatcggtgagagcttgctaggacggatcactgcagg

SEQ ID NO: 12 - DNA, 50 bp, Artificial Sequence
  synthetic polynucleotide
tccagaagcttccggtagcttaaggtagccaaatgcctcgtcatctaatt

SEQ ID NO: 13 - DNA, 50 bp, Artificial Sequence
  synthetic polynucleotide
aattagatgacgaggcatttggctaccttaagctaccggaagcttctgga

SEQ ID NO: 14 - DNA, 25 bp, Artificial Sequence
  synthetic polynucleotide
aattagatgacgaggcatttggcta

SEQ ID NO: 15 - DNA, 25 bp, Artificial Sequence
  synthetic polynucleotide
ccttaagctaccggaagcttctgga

SEQ ID NO: 16 - DNA, 50 bp, Artificial Sequence
  synthetic polynucleotide
gctcgagtcgtacttatcggtgagacgctgctaccggaagcttctggacc

SEQ ID NO: 17 - DNA, 50 bp, Artificial Sequence
  synthetic polynucleotide
tggcatgatgatccggcgatgaaaaccttaagctaccggaagcttctgga

SEQ ID NO: 18 - DNA, 50 bp, Artificial Sequence
  synthetic polynucleotide
aattagatgacgaggcatttggctatctcaccgataagtacgactcgagc

SEQ ID NO: 19 - DNA, 25 bp, Artificial Sequence
  synthetic polynucleotide
tggcatgatgatccggcgatgaaaa

SEQ ID NO: 20 - RNA, 25 nt, Artificial Sequence
  synthetic polynucleotide
uggcaugaugauccggcgaugaaaa

SEQ ID NO: 21 - DNA, 50 bp, Artificial Sequence
  synthetic polynucleotide
aaattaaaattatgcgtatcgccccccttaagctaccggaagcttctgga

SEQ ID NO: 22 - RNA, 25 nt, Artificial Sequence
  synthetic polynucleotide
ggggcgauacgcauaauuuuaauuu

SEQ ID NO: 23 - DNA, 50 bp, Artificial Sequence
  synthetic polynucleotide
tggcatgatgatccggcgatgaaaaggggcgatacgcataattttaattt

SEQ ID NO: 24 - DNA, 25 bp, Artificial Sequence
  synthetic polynucleotide
ggggcgatacgcataattttaattt

SEQ ID NO: 25 - DNA, 72 bp, Artificial Sequence
  synthetic polynucleotide
tccagaagcttccggtagcttaaggtagccaaatgcctcgtcatctaattagtgacgcgcatgaatggatta

SEQ ID NO: 26 - DNA, 72 bp, Artificial Sequence
  synthetic polynucleotide
taatccattcatgcgcgtcactaattagatgacgaggcatttggctattttcatcgccggatcatcatgcca

SEQ ID NO: 27 - DNA, 108 bp, Artificial Sequence
  synthetic polynucleotide
gctctgaatgtcaacgtgaagaaattcaagcaagcgcgggtaaacggcgggagtaactatgactctcttaaggtagggtccagaagcttccggtagcagcgagagcgg

SEQ ID NO: 28 - DNA, 60 bp, Artificial Sequence
  synthetic polynucleotide
ccgctctcgctgctaccggaagcttctggaccctattttcatcgccggatcatcatgcca

SEQ ID NO: 29 - DNA, 98 bp, Artificial Sequence
  synthetic polynucleotide
aaattaaaattatgcgtatcgccccccttaagagagtcatagttactcccgccgtttacccgcgcttgcttgaatttcttcacgttgacattcagagc

SEQ ID NO: 30 - DNA, 120 bp, Artificial Sequence
  synthetic polynucleotide
gctctgaatgtcaacgtgaagaaattcaagcaagcgcgggtaaacggcgggagtaactatgactctcttaaggtagccaaatgcctcgtcatctaattagtgacgcgcatgaatggatta

SEQ ID NO: 31 - protein, 78 aa, Artificial Sequence
  synthetic polypeptide
ESTRTPTSTKWIRERCAQITGRDFVQFVHTHINALPSRIRGSRGRRGGGESSLTCRAGCKVRETTAHILQQCHRTHGG

SEQ ID NO: 32 - protein, 75 aa, Artificial Sequence
  synthetic polypeptide
EGGHYGPQHGWVSQPTRLLTGKEYMDGIRLRINALPTKSRTTRGRHELERQCRAGCDAPETTNHIMQKCYRSHGR

SEQ ID NO: 33 - protein, 75 aa, Artificial Sequence
  synthetic polypeptide
EAERFAPQHGWVSQPTRLLTGKEYTDGIKLRINALPSRSRTTRGRHELGRRCRAGCDAPETTNHILQQCYRTHGR

SEQ ID NO: 34 - protein, 75 aa, Artificial Sequence
  synthetic polypeptide
EARHYGPQHGWVSQPTRLLTGKEYLHGVKLRINALPSKSRTTRGRHELERRCRAGCDAPETTNHILQKCYRTHGR

SEQ ID NO: 35 - protein, 70 aa, Artificial Sequence
  synthetic polypeptide
EAGHWGPQHGWVNQPTRLLSGKEYIDGIRLRINALPTKSRTTRGRHVGEGCDAPETTNHIMQKCYQSHGR

SEQ ID NO: 36 - protein, 75 aa, Artificial Sequence
  synthetic polypeptide
EAGLFRPQHGWVCQPTRLLTGQDYRNGIKLRINALPSRSRTTRGRNELERQCRAGCDAPETTNHILQNCYRTHGR

SEQ ID NO: 37 - protein, 74 aa, Artificial Sequence
  synthetic polypeptide
ESSKVPQQHRWATGGSRFLTGRDFINSIKLRINTLPTLSRTLRGREGNRMCRGGCYNVETLHHVLQVCHRTNGT

SEQ ID NO: 38 - protein, 74 aa, Artificial Sequence
  synthetic polypeptide
EAGRVKGIHSWTCDGRMVMTGQAFCEALKIRINALPCLSRYNRGTEKPRECRAGCKTTESLNHVLQVCPRTHDM

SEQ ID NO: 39 - protein, 76 aa, Artificial Sequence
  synthetic polypeptide
DTRKAEASYSWIRDIHVAIPASVWIKYHHTRINALPTLMRMSRGRRTNGNALCRAGCGLPETLYHVVQQCPRTHGG

SEQ ID NO: 40 - protein, 77 aa, Artificial Sequence
  synthetic polypeptide
LFKGSRSANHWLPRPVGMKPHHWVKCLAMRANVYPTKRGLSRGNLSKNKDSAKCRGCTSMRETLCHLSGQCPKLKSM

SEQ ID NO: 41 - protein, 74 aa, Artificial Sequence
  synthetic polypeptide
EFFGDKTGNAWLYNPEMLRPSRYLDALKLRTNTYGTKAALHRAKRDIDINCRRCGVQVETLGHILGLCTHTKNK

SEQ ID NO: 42 - protein, 74 aa, Artificial Sequence
  synthetic polypeptide
GFFRNRVANGWLRKPAGFKERHYIAALQLRACVYPTLEFQQRGRSKAGAACRRCSSRLESSSHILGKCPAVQGA

SEQ ID NO: 43 - protein, 75 aa, Artificial Sequence
  synthetic polypeptide
TIAAVSSVNRYWMIEDNLKSGNKILVWKAMAGAIPTKINLSRGVADQTLKKCRRCGLTAETDGHILAGCHTSSDA

SEQ ID NO: 44 - protein, 74 aa, Artificial Sequence
  synthetic polypeptide
VFSSSPLTNQWINGEIKTMTTKTYINSIKLRTNTLETRVTTSRGLNIIKTCRRCHVADESLMHVLQCCSSTKGL